(19) United States
(12) Patent Application Publication
(10) Pub. No.: US 2018/0298428 A1
(43) Pub. Date: Oct. 18, 2018

(54) NUCLEIC ACID DETECTION AND QUANTIFICATION METHOD AND COMPOSITIONS

(71) Applicants: Microbial Discovery Group LLC, Franklin, WI (US); JBS United ,Inc., Sheridan, IN (US)

(72) Inventors: Michael R KING, Oak Creek, WI (US); Sona SON, Franklin, WI (US); Joel D SPENCER, Westfield, IN (US); Amy LANGE, Greendale, WI (US); Jessica EDWARD, Kenosha, WI (US); Ardean VELDKAMP, Oostburg, WI (US)

(73) Assignees: Microbial Discovery Group LLC, Franklin, WI (US); JBS United, Inc., Sheridan, IN (US)

(21) Appl. No.: 15/570,559

(22) PCT Filed: Apr. 29, 2016
(86) PCT No.: PCT/US2016/030223

§ 371 Date: Oct. 30, 2017

Related U.S. Application Data
(60) Provisional application No. 62/165,127, filed on May 21, 2015.
(60) Provisional application No. 62/155,778, filed on May 1, 2015.

(51) Int. Cl. C12Q 1/689; C12Q 1/6851; C12Q 1/6806; C12Q 1/6809; C12M 1/00

(52) U.S. Cl. CPC C12Q 1/689; C12Q 1/6851; C12Q 1/6806; C12Q 2565/625; C12M 45/22; C12Q 2531/113; C12Q 2545/113; C12Q 1/6809

(57) ABSTRACT

This invention relates to a method of detecting a gene. The invention also relates to a method of determining the Amplification expression level of a gene. The invention also relates to compositions for use in these methods.

### CROSS REFERENCE TO RELATED U.S. PATENT APPLICATIONS

This application claims priority under 35 U.S.C. § 119(e) to U.S. Provisional Patent Application No. 62/155,778, filed May 1, 2015 and U.S. Provisional Patent Application No. 62/165,127, filed May 21, 2015, the disclosures of which are incorporated herein by reference.

### TECHNICAL FIELD

This invention relates to a method of detecting a gene. The invention also relates to a method of determining the expression level of a gene. The invention also relates to compositions for use in these methods.

### BACKGROUND AND SUMMARY

Numerous microorganisms live among humans, domestic animals and wildlife. The majority of these species exist in a beneficial or symbiotic manner, however, some species are capable of producing toxins or can be detrimental in other ways which can consequently result in disease, or other detrimental effects, with either subclinical or clinic indicators for humans, domestic animals, and wildlife. Accordingly, a process is needed that allows detection and/or quantification of microorganisms (e.g., pathogenic microorganisms) or detrimental substances produced by microorganisms (e.g., toxins or other virulence factors) in a specific, sensitive, and time and cost efficient manner where samples of the microorganisms are transported over long distances. Traditional methods require mailing a sample to a lab (making it susceptible to being compromised from temperature and other environmental conditions), plating the sample on selective media to isolate individual colonies, using endpoint polymerase chain reaction (PCR) to amplify any gene of interest, and running an electrophoresis gel to determine the size and purity of the nucleic acid. These methods allow determination of the presence or absence of nucleic acids, but do not allow for quantification. More recently, quantitative real-time PCR has been developed to quantify the amount of a nucleic acid using fluorescence technology. Although this process advances the detection system, it does not address the labor intensiveness and price associated with using selective plating methods to recover microorganisms.

Applicants have developed a method that 1) eliminates the need for labor intensive and costly selective plating methods to recover microorganisms, and 2) is capable of quantifying microorganisms and/or their specific genes (e.g., toxin or virulence-associated genes). The method also allows for determination of total microbial load and for stabilization of nucleic acids transported over long distances, for example, at room temperature. This method, and the compositions therefor, allow for an advanced process that is more rapid, more sensitive, and provides more accurate results than with previous methods.

Several embodiments of the invention are also described by the following enumerated clauses:

1. A method of quantifying the expression level of a gene from a microorganism, the method comprising the steps of:

recovering the nucleic acid from a sample stabilized on a card,

amplifying the nucleic acid; and

quantifying the expression level of the gene, wherein a forward primer, and a reverse primer are used for the amplification.

2. The method of clause 1 further comprising the step of hybridizing a probe to the nucleic acid to specifically identify the gene.

3. The method of clause 1 or 2 wherein the reverse primer comprises a sequence selected from the group consisting of SEQ ID NO: 6 and SEQ ID NO: 8.

4. The method of any one of clauses 1 to 3 wherein the nucleic acid is DNA.

5. The method of any one of clauses 1 to 3 wherein the nucleic acid is RNA.

6. The method of any one of clauses 1 to 5 wherein the nucleic acid is amplified using PCR.

7. The method of clause 6 wherein the PCR is reverse transcription PCR.

8. The method of clause 6 wherein the PCR is reverse transcription-quantitative PCR.

9. The method of any one of clauses 2 to 8 wherein the probe is fluorescently labeled.

10. The method of any one of clauses 1 to 9 wherein the primer is fluorescently labeled.

11. The method of any one of clauses 1 to 10 wherein the microorganism is selected from the group consisting of Vibrio harveyi, Vibrio campbellii, Vibrio fluvialis, and Vibrio parahaemolyticus.

12. The method of any one of clauses 1 to 10 wherein the microorganism is selected from the group consisting of Clostridium perfringens, Campylobacter jejuni, and Campylobacter coli.

13. The method of any one of clauses 1 to 12 wherein the sample is a sample from an animal.

14. The method of any one of clauses 1 to 13 wherein the sample is an aquatic sample.

15. The method of clause 14 wherein the aquatic sample is from a fish hatchery.

16. The method of clause 14 wherein the aquatic sample is from a shrimp pond.

17. The method of any one of clauses 1 to 13 wherein the sample is an agricultural sample.

18. The method of clause 17 wherein the agricultural sample is from animal litter.

19. The method of clause 17 wherein the agricultural sample is a swab from a swine or a poultry species.

20. The method of any one of clauses 1 to 19 wherein the gene is a gene encoding a toxin.

21. The method of any one of clauses 1 to 20 wherein the gene is a gene of a bacterial species.

22. The method of any one of clauses 1 to 20 wherein the gene is a gene of a viral species.

23. The method of any one of clauses 1 to 11 or 13 to 21 wherein the gene is a hemolysin (hly) gene.

24. The method of any one of clauses 1 to 10 or 12 to 21 wherein the gene is a Clostridium perfringens enterotoxin (cpe) gene.

25. The method of any one of clauses 1 to 10 or 12 to 21 wherein the gene is a Clostridium perfringens beta toxin (cpb) gene.

26. The method of any one of clauses 1 to 25 wherein the nucleic acid is stabilized on the card for a period of time to allow transportation overseas.

27. The method of any one of clauses 1 to 26 wherein the nucleic acid is stabilized on the card for a period of time to allow transportation over greater than 1000 miles.

28. The method of any one of clauses 1 to 26 wherein the nucleic acid is stabilized on the card for a period of time to allow transportation over greater than 2000 miles.

29. The method of any one of clauses 1 to 26 wherein the nucleic acid is stabilized on the card for a period of time to allow transportation over greater than 3000 miles.

30. The method of any one of clauses 1 to 26 wherein the nucleic acid is stabilized on the card for a period of time to allow transportation over greater than 5000 miles.

31. The method of any one of clauses 1 to 30 wherein the card is a WHATMAN® FTA® Card.

32. The method of any one of clauses 1 to 31 wherein the reverse primer has the sequence of SEQ ID NO: 6.

33. The method of any one of clauses 1 to 31 wherein the reverse primer has the sequence of SEQ ID NO: 8.

34. A kit comprising at least one primer pair, wherein the at least one primer pair comprises a forward primer and a reverse primer, and wherein the reverse primer has a sequence selected from the group consisting of SEQ ID NO: 6 and SEQ ID NO: 8.

35. The kit of clause 34 wherein the at least one primer pair is fluorogenic.

36. The kit of clause 35 wherein the at least one primer pair is fluorescently labeled.

37. The kit of any one of clauses 34 to 36 wherein the forward primer has a sequence selected from the group consisting of SEQ ID NO: 1, SEQ ID NO: 3, SEQ ID NO: 5, and SEQ ID NO. 7.

38. The kit of any one of clauses 34 to 37 wherein the reverse primer has the sequence of SEQ ID NO. 6.

39. The kit of any one of clauses 34 to 37 wherein the reverse primer has the sequence of SEQ ID NO. 8.

40. The kit of any one of clauses 34 to 39 further comprising a reverse transcriptase.

41. The kit of any one of clauses 34 to 40 further comprising a DNA polymerase.

42. The kit of any one of clauses 34 to 41 further comprising dNTPs.

43. The kit of any one of clauses 34 to 42 further comprising a fluorogenic probe.

44. The method of any one of clauses 1 to 10 or 13 to 21 wherein microorganism is selected from the group consisting of swine enterotoxigenic E. coli (ETEC), avian pathogenic E. coli (APEC), attaching and effacing E. coli (EAEC), enterohaemorrhagic E. coli (EHEC), and shiga toxin-producing E. coli (STEC).

45. The method of clause 44 wherein the ETEC is an antigenic type selected from the group consisting of K88, F18, F41, 987P, and K99.

46. The method of any one of clauses 1 to 33, 44, or 45 wherein reverse transcription-PCR and endpoint PCR are performed.

47. The method of clause 6, wherein the PCR is quantitative PCR.

### BRIEF DESCRIPTION OF THE DRAWINGS

FIG. 1A shows the amplification analysis for a qPCR reaction.

FIG. 1B shows the standard curve analysis for a qPCR reaction.

FIG. 2A shows relative quantities of Clostridium perfringens from poultry litter samples of pen 43.

FIG. 2B shows relative quantities of Clostridium perfringens from poultry litter samples of pen 41.

FIG. 2C shows relative quantities of Clostridium perfringens from poultry litter samples of pen 14.

FIG. 2D shows relative quantities of Clostridium perfringens from poultry litter samples of pen 44.

FIG. 3A shows the amplification analysis for a qPCR reaction using FTA cards and genomic DNA controls.

FIG. 3B shows the standard curve analysis for a qPCR reaction using FTA cards and genomic DNA controls.

FIG. 4A shows the relative quantity levels for the 16S reference gene, along with the tcdA and tcdB genes of interest.

FIG. 4B shows the expression levels for tcdA and tcdB genes of interest normalized to the 16S reference gene.

FIG. 5A shows the standard curve analysis for a qPCR reaction using FTA cards and genomic DNA control samples.

FIG. 5B shows the amplification analysis for a qPCR reaction using FTA cards and genomic DNA control samples.

FIG. 6A shows the amplification analysis for a qPCR reaction using FTA card controls and samples.

FIG. 6B shows the standard curve analysis for a qPCR reaction using FTA card controls and samples.

FIG. 7A shows quantification data based on a standard curve.

FIG. 7B shows quantification data based on a standard curve.

FIG. 8 shows the correlation between total bacterial load and total Vibrio load in the samples tested.

FIG. 9A shows an RNA extraction comparative analysis for Vibrio spp. (hyl).

FIG. 9B shows a storage time and temperature comparative analysis for Vibrio spp. (hyl).

FIG. 10A shows aquaculture pond analysis of bacterial counts for V. campbellii, V. harveyi, and total Vibrio.

FIG. 10B shows aquaculture pond analysis of bacterial load for V. campbellii, V. harveyi, and other Vibrio.

### DETAILED DESCRIPTION OF ILLUSTRATIVE EMBODIMENTS

In one embodiment, a method is provided for quantifying the expression level of a gene from a microorganism. The method comprises the steps of recovering the nucleic acid from a sample stabilized on a card, amplifying the nucleic acid, and quantifying the expression level of the gene, wherein a forward primer, and a reverse primer are used for the amplification. In another embodiment, a kit is provided. The kit comprises at least one primer pair, wherein the at least one primer pair comprises a forward primer and a reverse primer, and wherein the reverse primer has a sequence consisting of SEQ ID NO: 6 or SEQ ID NO:8. In one embodiment, the kit further comprises a fluorogenic probe. In another embodiment, the kit further comprises a card (e.g., an FTA card).

As used herein, the term “nucleic acid” can mean, for example, DNA, RNA, including mRNA, an siRNA, an iRNA, or a microRNA.

As used herein, the term “card” can means any tangible medium (e.g., paper) that has been chemically modified or chemically treated to stabilize nucleic acids. An example of a “card” for use in the method described herein is a Whatman® FTA® Card.

Several embodiments of the invention are described in the Summary section of this patent application and each of the embodiments described in this Detailed Description section of the application applies to the embodiments described in the Summary, including the embodiments described by the enumerated clauses below. In any of the various embodiments described herein, the following features in the enumerated clauses may be present where applicable, providing additional embodiments of the invention. For all of the embodiments, any applicable combination of embodiments is also contemplated.

1. A method of quantifying the expression level of a gene from a microorganism, the method comprising the steps of:

recovering the nucleic acid from a sample stabilized on a card,

amplifying the nucleic acid; and

quantifying the expression level of the gene, wherein a forward primer, and a reverse primer are used for the amplification.

2. The method of clause 1 further comprising the step of hybridizing a probe to the nucleic acid to specifically identify the gene.

3. The method of clause 1 or 2 wherein the reverse primer comprises a sequence selected from the group consisting of SEQ ID NO: 6 and SEQ ID NO: 8.

4. The method of any one of clauses 1 to 3 wherein the nucleic acid is DNA.

5. The method of any one of clauses 1 to 3 wherein the nucleic acid is RNA.

6. The method of any one of clauses 1 to 5 wherein the nucleic acid is amplified using PCR.

7. The method of clause 6 wherein the PCR is reverse transcription PCR.

8. The method of clause 6 wherein the PCR is reverse transcription-quantitative PCR.

9. The method of any one of clauses 2 to 8 wherein the probe is fluorescently labeled.

10. The method of any one of clauses 1 to 9 wherein the primer is fluorescently labeled.

11. The method of any one of clauses 1 to 10 wherein the microorganism is selected from the group consisting of Vibrio harveyi, Vibrio campbellii, Vibrio fluvialis, and Vibrio parahaemolyticus.

12. The method of any one of clauses 1 to 10 wherein the microorganism is selected from the group consisting of Clostridium perfringens, Campylobacter jejuni, and Campylobacter coli.

13. The method of any one of clauses 1 to 12 wherein the sample is a sample from an animal.

14. The method of any one of clauses 1 to 13 wherein the sample is an aquatic sample.

15. The method of clause 14 wherein the aquatic sample is from a fish hatchery.

16. The method of clause 14 wherein the aquatic sample is from a shrimp pond.

17. The method of any one of clauses 1 to 13 wherein the sample is an agricultural sample.

18. The method of clause 17 wherein the agricultural sample is from animal litter.

19. The method of clause 17 wherein the agricultural sample is a swab from a swine or a poultry species.

20. The method of any one of clauses 1 to 19 wherein the gene is a gene encoding a toxin.

21. The method of any one of clauses 1 to 20 wherein the gene is a gene of a bacterial species.

22. The method of any one of clauses 1 to 20 wherein the gene is a gene of a viral species.

23. The method of any one of clauses 1 to 11 or 13 to 21 wherein the gene is a hemolysin (hly) gene.

24. The method of any one of clauses 1 to 10 or 12 to 21 wherein the gene is a Clostridium perfringens enterotoxin (cpe) gene.

25. The method of any one of clauses 1 to 10 or 12 to 21 wherein the gene is a Clostridium perfringens beta toxin (cpb) gene.

26. The method of any one of clauses 1 to 25 wherein the nucleic acid is stabilized on the card for a period of time to allow transportation overseas.

27. The method of any one of clauses 1 to 26 wherein the nucleic acid is stabilized on the card for a period of time to allow transportation over greater than 1000 miles.

28. The method of any one of clauses 1 to 26 wherein the nucleic acid is stabilized on the card for a period of time to allow transportation over greater than 2000 miles.

29. The method of any one of clauses 1 to 26 wherein the nucleic acid is stabilized on the card for a period of time to allow transportation over greater than 3000 miles.

30. The method of any one of clauses 1 to 26 wherein the nucleic acid is stabilized on the card for a period of time to allow transportation over greater than 5000 miles.

31. The method of any one of clauses 1 to 30 wherein the card is a WHATMAN® FTA® Card.

32. The method of any one of clauses 1 to 31 wherein the reverse primer has the sequence of SEQ ID NO: 6.

33. The method of any one of clauses 1 to 31 wherein the reverse primer has the sequence of SEQ ID NO: 8.

34. A kit comprising at least one primer pair, wherein the at least one primer pair comprises a forward primer and a reverse primer, and wherein the reverse primer has a sequence selected from the group consisting of SEQ ID NO: 6 and SEQ ID NO: 8.

35. The kit of clause 34 wherein the at least one primer pair is fluorogenic.

36. The kit of clause 35 wherein the at least one primer pair is fluorescently labeled.

37. The kit of any one of clauses 34 to 36 wherein the forward primer has a sequence selected from the group consisting of SEQ ID NO: 1, SEQ ID NO: 3, SEQ ID NO: 5, and SEQ ID NO. 7.

38. The kit of any one of clauses 34 to 37 wherein the reverse primer has the sequence of SEQ ID NO. 6.

39. The kit of any one of clauses 34 to 37 wherein the reverse primer has the sequence of SEQ ID NO. 8.

40. The kit of any one of clauses 34 to 39 further comprising a reverse transcriptase.

41. The kit of any one of clauses 34 to 40 further comprising a DNA polymerase.

42. The kit of any one of clauses 32 to 41 further comprising dNTPs.

43. The kit of any one of clauses 34 to 42 further comprising a fluorogenic probe.

44. The method of any one of clauses 1 to 10 or 13 to 21 wherein microorganism is selected from the group consisting of swine enterotoxigenic E. coli (ETEC), avian pathogenic E. coli (APEC), attaching and effacing E. coli (EAEC), enterohaemorrhagic E. coli (EHEC), and shiga toxin-producing E. coli (STEC).

45. The method of clause 44 wherein the ETEC is an antigenic type selected from the group consisting of K88, F18, F41, 987P, and K99.

46. The method of any one of clauses 1 to 33, 44, or 45 wherein reverse transcription-PCR and endpoint PCR are performed.

47. The method of clause 6, wherein the PCR is quantitative PCR.

The methods and compositions for detection and/or quantification of microorganisms or their genes (e.g., toxin or virulence genes) are specific and sensitive. In various embodiments, the microorganism that is detected or for which the level of expression of a gene is quantified may be any microorganism that infects an animal. In various embodiments, the microorganism may include such pathogens as bacteria, including gram-negative or gram-positive cocci or bacilli, fungi, viruses, including DNA and RNA viruses, mycoplasma, and parasites.

In one embodiment, the microorganism is a bacterium. In one aspect of this embodiment, the bacteria may include, but are not limited to, Acetobacter, Actinomyces, Agrobacterium, Anaplasma, Azorhizobium, Azotobacter, Bacillus, Bacteroides, Bartonella, Bordetella, Burkholderia, Campylobacter, Chlamydia, Chlamydophila, Clostridium, Corynebacterium, Coxiella, Ehrlichia, Enterobacter, Enterococcus, Escherichia, Francisella, Fusobacterium, Gardnerella, Haemophilus, Helicobacter, Klebsiella, Lactobacillus, Legionella, Listeria, Methanobacterium, Microbacterium, Micrococcus, Moraxella, Mycobacterium, Mycoplasma, Neisseria, Pasteurella, Peptostreptococcus, Porphyromonas, Prevotella, Pseudomonas, Rhizobium, Rickettsia, Rochalimaea, Rochalimaea, Rothia, Salmonella, Serratia, Shigella, Staphylococcus, Stenotrophomonas, Streptococcus, Treponema, Vibrio, Wolbachia, or Yersinia species.

In another aspect, the microorganism is selected from the group consisting of swine enterotoxigenic E. coli (ETEC), avian pathogenic E. coli (APEC), attaching and effacing E. coli (EAEC), enterohaemorrhagic E. coli (EHEC), and shiga toxin-producing E. coli (STEC). In yet another illustrative aspect, the enterotoxigenic E. coli (ETEC) is an antigenic type selected from the group consisting of K88, F18, F41, 987P, and K99. The avian pathogenic E. coli (APEC) produces toxins such as, but not limited to, labile toxin (LT), stable toxin A (StA), stable toxin B (StB), and verotoxin (shiga-like toxin, SLT). Enterohaemorrhagic E. coli (EHEC) is a bacterium that can cause severe foodborne disease. Shiga toxin-producing E. coli (STEC) is a bacterial pathotype that is most commonly described in the media as the cause of foodborne disease outbreaks.

In another embodiment, the microorganism is a virus. In one aspect, the viruses may include, but are not limited to, DNA viruses such as papilloma viruses, parvoviruses, adenoviruses, herpesviruses and vaccinia viruses, and RNA viruses, such as arenaviruses, coronaviruses, rhinoviruses, respiratory syncytial viruses, influenza viruses, picornaviruses, paramyxoviruses, reoviruses, retroviruses, and rhabdoviruses.

Examples of fungi include fungi that grow as molds or are yeastlike, including, for example, fungi that cause diseases such as ringworm, histoplasmosis, blastomycosis, aspergillosis, cryptococcosis, sporotrichosis, coccidioidomycosis, paracoccidio-idomycosis, and candidiasis.

Exemplary parasites include, but are not limited to, somatic tapeworms, blood flukes, tissue roundworms, ameba, and Plasmodium, Trypanosoma, Leishmania, and Toxoplasma species. In various aspects of the microorganism embodiments described in the preceding paragraphs, the expression of any gene expressed by any of these microorganisms can be quantified using the method described herein. In various embodiments, the gene can be a gene encoding a toxin or a virulence factor.

In another embodiment, the sample that is tested can be any sample from any animal. As used herein the word “animal” means a human, a domestic animal (e.g., a canine or a feline species), a laboratory animal, an agricultural animal, or wildlife, or any other type of animal. As used herein, an agricultural animal may include any animal that is raised for personal use (e.g., for providing food, fuel, etc.) or for profit. In yet another embodiment, a domestic animal may include any animal that is kept or raised for companionship purposes (e.g., a dog or a cat). Accordingly, in various embodiments, the invention can be applied to samples from animals including, but not limited to, humans (e.g., a human patient), laboratory animals such rodents (e.g., mice, rats, hamsters, etc.), rabbits, monkeys, chimpanzees, domestic animals such as dogs, cats, and rabbits, agricultural animals such as cows, horses, ponies, pigs, sheep, goats, fish, crustaceans, shrimp, chickens, turkeys, pheasants, quails, ostriches, and ducks, and wild animals, for example, wild animals in captivity, such as bears, pandas, lions, tigers, leopards, elephants, zebras, giraffes, gorillas, dolphins, and whales.

In one aspect, the agricultural animal from which a sample is taken may include a bovine species (e.g., cattle and bison), an equine species (e.g., horses, ponies, and donkeys), an ovine species (e.g., sheep), a caprine species (e.g., goats), rabbits, and poultry (e.g., chickens, turkeys, pheasant, ducks, ostriches, emu, quail, and geese).

In other embodiments, the sample may be from the environment, including the environment of an animal. The sample may be an aquatic sample, such as a water sample from a fish hatchery, a sample from a shrimp pond, a sample from an animal's drinking water, etc. In another aspect, the sample may be an agricultural sample, such as a sample from animal litter, or any other agricultural environmental sample, a swab from the intestinal tract of an agricultural animal (e.g., a swine or poultry species), a swab from the nasal tract of an agricultural animal, a swab from the skin of an agricultural animal, a swab from the ear of an agricultural animal, a swab from the eye of an agricultural animal, a urine sample from an agricultural animal, a nasal secretion sample from an agricultural animal, a bronchial lavage from an agricultural animal, a spinal fluid sample of an agricultural animal, a pleural fluid sample from an agricultural animal, a synovial fluid sample from an agricultural animal, a gastric secretions sample from an agricultural animal, a sample from feces of an agricultural animal, or a serum or plasma sample from an agricultural animal.

In various illustrative embodiments, samples from humans that can be tested for the presence of microorganism or their genes or from which gene expression can be quantified, include, but are not limited to, urine, nasal secretions, nasal washes, inner ear fluids, bronchial lavages, bronchial washes, alveolar lavages, spinal fluid, bone marrow aspirates, sputum, pleural fluids, synovial fluids, pericardial fluids, peritoneal fluids, saliva, tears, gastric secretions, stool, reproductive tract secretions, such as seminal fluid, lymph fluid, and whole blood, serum, or plasma. In another embodiment, the samples can be prepared for testing as described herein using the types of cards described herein. In various embodiments, these samples can include tissue biopsies. As used herein, the term “tissue” includes, but is not limited to, biopsies, autopsy specimens, cell extracts, tissue sections, aspirates, tissue swabs, and fine needle aspirates. In another embodiment, the sample can be any environmental sample.

The samples tested in accordance with the method described herein can be stabilized (e.g., the nucleic acid can be stabilized) on a card (e.g., a Whatman® FTA® Card) for a period of time to allow transportation overseas or over a long distance. In various embodiments, the nucleic acid is stabilized on the card for a period of time to allow transportation over greater than 1000 miles, greater than 2000 miles, greater than 3000 miles, greater than 4000 miles, greater than 5000 miles, greater than 6000 miles, greater than 7000 miles, greater than 8000 miles, greater than 9000 miles, or greater than 10000 miles. In other embodiments, the nucleic acid is stabilized on the card for a period of time to allow transportation over greater than 10 miles, over greater than 20 miles, over greater than 30 miles, over greater than 40 miles, over greater than 50 miles, over greater than 60 miles, over greater than 70 miles, over greater than 80 miles, over greater than 90 miles, over greater than 100 miles, over greater than 200 miles, over greater than 300 miles, over greater than 400 miles, over greater than 500 miles, over greater than 600 miles, over greater than 700 miles, over greater than 800 miles, or over greater than 900 miles. In various embodiments, the nucleic acid can be stabilized on the card for a period of time selected from the group consisting of 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 week, 2 weeks, 3 weeks, 4 weeks, 5 weeks, 6 weeks, 7 weeks, 8 weeks, 9 weeks, 10 weeks, 1 month, 2 months, 3 months, 4 months, 5 months, 6 months, 7 months, 8 months, 9 months, 10 months, 11 months, and 12 months, or greater than any of these time periods.

The methods and compositions described herein can be used to detect and/or quantify microorganisms and/or their genes (e.g., the level of expression of a gene). In one illustrative embodiment, a method is provided of quantifying the expression level of a gene from a microorganism. The method comprises the steps of recovering a nucleic acid from a sample on a card, amplifying the nucleic acid, and quantifying the expression level of the gene. A reverse primer and a forward primer are used in the amplification step. The method can further comprise hybridizing a probe to the nucleic acid to specifically identify the gene.

In one aspect, the methods described herein can be more sensitive than endpoint PCR, for example at least 50-fold, at least 60-fold, at least 70-fold, at least 80-fold, at least 90-fold, or at least 100-fold more sensitive. In another embodiment, the methods described herein can detect from 1-3, from 1-5, from 1-10, from 1-20, from 1-30, from 1-40, from 1-50, from 1-60, from 1-70, from 1-80, from 1-90, or from 1-100 cell equivalents per PCR tube. Thus, the methods described herein are surprisingly more sensitive than other assays.

In some embodiments, real-time PCR-based methods can be used to amplify the nucleic acid and to detect and/or quantify the microorganism and/or the gene expressed by the microorganism by hybridization of a probe to the nucleic acid. PCR is described in U.S. Pat. Nos. 4,683,202 and 4,800,159, incorporated herein by reference, and methods for PCR are well-known in the art. Real-time PCR can combine amplification and simultaneous probe hybridization to achieve sensitive and specific detection and/or quantitation of microorganisms or the genes they express in real-time thereby providing instant detection and/or quantification. In this embodiment, the time to detect and/or quantify the microorganism or the gene expression is greatly reduced. Real-time PCR can be conducted according to methods well-known in the art. Reverse transcription PCR is a highly sensitive technique for the detection and quantification of mRNA that comprises the synthesis of cDNA from RNA by reverse transcription and the amplification of a specific cDNA by PCR. In one aspect, reverse transcription quantitative PCR quantitatively measures the amplification of the cDNA by using fluorescent probes. Real-time PCR and reverse transcription quantitative PCR can also be performed without probes.

Exemplary probes and primers and their target nucleic acids that can be used in accordance with the invention are shown below. Forward primers and reverse primers are shown and are well-known terms in the art.

TABLE 1
Primers

| | |
|---|---|
| Sequence Description | Sequence |

Vibrio campbellii hly

| | |
|---|---|
| Forward Primer (SEQ ID NO: 1) | CTATTGGTGGAACGCAC |
| Reverse Primer (SEQ ID NO: 2) | GTATTCTGTCCATACAAAC |

Vibrio harveyi hly

| | |
|---|---|
| Forward Primer (SEQ ID NO: 3) | GAGTTCGGTTTCTTTCAAG |
| Reverse Primer (SEQ ID NO: 4) | TGTAGTTTTTCGCTAATTTC |

Vibrio campbellii hly (short amplicon)

| | |
|---|---|
| Forward Primer (SEQ ID NO: 5) | CTATTGGTGGAACGCAC |
| Reverse Primer (SEQ ID NO: 6) | CAGCGAAGTAGGTAATGTC |

Vibrio harveyi hly (short amplicon)

| | |
|---|---|
| Forward Primer (SEQ ID NO: 7) | GAGTTCGGTTTCTTTCAAG |
| Reverse Primer (SEQ ID NO: 8) | AAACGGTTATCGGCTG |

Vibrio 16S rRNA

| | |
|---|---|
| Forward Primer (SEQ ID NO: 9) | GGCGTAAAGCGCATGCAGGT |
| Reverse Primer (SEQ ID NO: 10) | GAAATTCTACCCCCCTCTACAG |

Bacterial 16S rRNA

| | |
|---|---|
| Forward Primer (SEQ ID NO: 11) | 27F: |
| | AGAGTTTGATCMTGGCTCAG |
| Reverse Primer (SEQ ID NO: 12) | 1492R: |
| | GGTTACCTTGTTACGACTT |

In various embodiments described herein, the primers and probes used for amplification of the nucleic acid and for detection and/or quantification of microorganisms and/or their genes are oligonucleotides from about ten to about one hundred, more typically from about ten to about thirty or about six to about twenty-five base pairs long, but any suitable sequence length can be used. In illustrative embodiments, the primers and probes may be double-stranded or single-stranded, but the primers and probes are typically single-stranded. In another embodiment, the primers and probes described herein are capable of specific hybridization, under appropriate hybridization conditions (e.g., appropriate buffer, ionic strength, temperature, formamide, and MgCl₂ concentrations), to a region of the target nucleic acid. In another aspect, the primers and probes described herein are designed based on having a melting temperature within a certain range, and substantial complementarity to the target nucleic acid. Methods for the design of primers and probes are described in Sambrook et al., “Molecular Cloning: A Laboratory Manual”, 3rd Edition, Cold Spring Harbor Laboratory Press, (2001), incorporated herein by reference.

In one illustrative embodiment, universal primers can be used to provide a method for determining the presence of a nucleic acid before conducting target-specific assays or for determining the level of a specific nucleic acid relative to total nucleic acid present. Exemplary bacterial universal primers can have the sequences:

### Example 16S Universal Bacterial Primers:

forward primer,
(SEQ ID NO. 13)
5′-GCGGATCCGCGGCCGCTGCAGAGTTTGATCCTGGCTCA G-3′
forward primer
(SEQ ID NO. 14)
5′-GCGGATCCTCTAGACTGCAGTGCCAGCAGCCGCGGTAA-3′
reverse primer
(SEQ ID NO. 15)
5′-GGCTCGAGCGGCCGCCCGGGTTACCTTGTTACGACTT-3′.

In various embodiments, the primers and probes described herein for use in PCR can be modified by substitution, deletion, truncation, and/or can be fused with other nucleic acid molecules wherein the resulting primers and probes hybridize specifically to the intended target nucleic acids and are useful in the methods described herein for amplification of the target nucleic acids. In one embodiment, derivatives can also be made such as phosphorothioate, phosphotriester, phosphoramidate, and methylphosphonate derivatives, that specifically bind to single-stranded DNA or RNA, for example (Goodchild, et al., Proc. Natl. Acad. Sci. 83:4143-4146 (1986)).

In one embodiment, the invention encompasses isolated or substantially purified nucleic acids. In another embodiment, an “isolated” or “purified” nucleic acid molecule is substantially free of other cellular material, or culture medium when produced by recombinant techniques, or substantially free of chemical precursors or other chemicals when chemically synthesized. Preferably, an “isolated” or “purified” nucleic acid is free of sequences that naturally flank the nucleic acid in the genomic nucleic acid from which it is derived. For example, in various embodiments, the isolated or purified nucleic acid can contain less than about 5 kb, 4 kb, 3 kb, 2 kb, 1 kb, 0.5 kb, or 0.1 kb of nucleotide sequences that naturally flank the nucleic acid in the genomic nucleic acids of the cell from which the nucleic acid is derived.

In one embodiment, nucleic acids complementary to the probes and primers described herein, and those that hybridize to the nucleic acids described herein or those that hybridize to their complements under highly stringent conditions are provided. In one aspect, “highly stringent conditions” means hybridization at 65° C. in 5×SSPE and 50% formamide, and washing at 65° C. in 0.5×SSPE. Conditions for high stringency, low stringency, and moderately stringent hybridization are described in Sambrook et al., “Molecular Cloning: A Laboratory Manual”, 3rd Edition, Cold Spring Harbor Laboratory Press, (2001), incorporated herein by reference. In some illustrative aspects, hybridization occurs along the full-length of the nucleic acid.

In one embodiment, nucleic acids having about 60%, about 70%, about 75%, about 80%, about 85%, about 90%, about 95%, 96%, 97%, 98%, 99%, or 99.5% homology to the probes and primers described herein can be used. Determination of percent identity or similarity between sequences can be done, for example, by using the GAP program (Genetics Computer Group, software; now available via Accelrys on http://www.accelrys.com), and alignments can be done using, for example, the ClustalW algorithm (VNTI software, InforMax Inc.). In one aspect, a sequence database can be searched using the nucleic acid sequence of interest. In another aspect, algorithms for database searching are typically based on the BLAST software (Altschul et al., 1990), and the percent identity can be determined along the full-length of the nucleic acid.

As used herein, the term “complementary” refers to the ability of purine and pyrimidine nucleotide sequences to associate through hydrogen bonding to form double-stranded nucleic acids. Guanine and cytosine, adenine and thymine, and adenine and uracil are complementary and can associate through hydrogen bonding resulting in the formation of double-stranded nucleic acids when two nucleic acids have “complementary” sequences. The complementary sequences can be DNA or RNA sequences. The complementary DNA or RNA sequences are referred to as a “complement.”

Techniques for synthesizing the probes and primers described herein are well-known in the art and include, but are not limited to, chemical syntheses and recombinant methods. Such techniques are described in Sambrook et al., “Molecular Cloning: A Laboratory Manual”, 3rd Edition, Cold Spring Harbor Laboratory Press, (2001), incorporated herein by reference. Primers and probes can also be made commercially (e.g., CytoMol, Sunnyvale, Calif. or Integrated DNA Technologies, Skokie, Ill.). Techniques for purifying or isolating the probes and primers described herein are well-known in the art. Exemplary techniques are described in Sambrook et al., “Molecular Cloning: A Laboratory Manual”, 3rd Edition, Cold Spring Harbor Laboratory Press, (2001), incorporated herein by reference. The primers and probes described herein can be analyzed by techniques known in the art, such as, for example, restriction enzyme analysis or sequencing, to determine if the sequence of the primers and probes is correct.

In various embodiments of the methods and compositions described herein, the probes and/or primers can be labeled, such as fluorescently labeled, radioactively labeled, or labeled with antigens, compounds such as biotin-avidin, colorimetric compounds, or other labeling agents known to those of skill in the art, to allow detection and quantification of amplified nucleic acids, such as by real-time reverse transcription quantitative PCR. In illustrative embodiments, the labels may include 6-carboxyfluorescein (FAM™), TET™ (tetrachloro-6-carboxyfluorescein), JOE™ (2,7,-dimethoxy-4,5-dichloro-6-carboxyfluorescein), VIC™, HEX (hexachloro-6-carboxyfluorescein), TAMRA™ (6-carboxy-N,N,N′,N′-tetramethylrhodamine), BHQ™, SYBR® Green, Alexa 350, Alexa 430, AlexaFluor 488, and AlexaFlour 647 (Molecular Probes, Eugene, Oreg.), AMCA, BODIPY 630/650, BODIPY 650/665, BODIPY-FL, BODIPY-R6G, BODIPY-TMR, BODIPY-TRX, Cascade Blue, Cy3, Cy5, Cy7, 6-FAM, fluorescein, rhodamine, phycoerythrin, biotin, ruthenium, DyLight fluorescent agents (DyLight 680, CW 800, trans-cyclooctene, tetrazine, methyltetrazine, and the like), Oregon Green, such as Oregon Green 488, Oregon Green 500, and Oregon Green 514, Pacific Blue, REG, Rhodamine Green, Rhodamine Red, ROX, and/or Texas Red. In one embodiment, the probes and/or primers can be fluorogenic (i.e., generate or enhance fluorescence). For example, the probes and/or primers may comprise a fluorescent label or a non-fluorescent molecule which is acted upon by a compound (e.g., an enzyme) to produce or enhance fluorescence.

The method embodiments described herein can provide methods of diagnosing infections. In one embodiment, humans in need of diagnosis of an infection can include a person exhibiting the symptoms of an infection, cancer patients, post-operative patients, transplant patients, wound-care patients, patients undergoing chemotherapy, immunosuppressed patients, and the like. In another embodiment, domestic animals, agricultural animals, laboratory animals, or wildlife in need of diagnosis of an infection can include any animal exhibiting the signs or symptoms of an infection.

In one embodiment, kits are provided. The kits are useful for detecting and/or quantitating microorganisms and/or their gene expression (e.g., the expression of a toxin or a virulence gene). In one aspect, the kit can contain the probes and/or primers described herein. In one aspect, the primers or the probe can be fluorogenic (e.g., fluorescently labeled). In another embodiment, the kit can also contain components for nucleic acid amplification, such as a heat stable DNA polymerase (e.g., Taq polymerase or Vent polymerase), buffers, MgCl₂, H₂O, dNTPs, a reverse transcriptase, and the like. In one embodiment, the reagents can remain in liquid form. In another embodiment, the reagents can be lyophilized. In another illustrative embodiment, the kits can also contain instructions for use.

In another embodiment, a kit comprising a nucleic acid with a sequence selected from the group consisting of SEQ ID NO: 1 to SEQ ID NO: 15 or a complement of a sequence selected from the group consisting of SEQ ID NO: 1 to SEQ ID NO: 15 is provided. In another embodiment, a kit comprising a nucleic acid with a sequence selected from the group consisting of SEQ ID NO: 6 and SEQ ID NO: 8 or a complement of a sequence selected from the group consisting of SEQ ID NO: 6 and SEQ ID NO: 8 is provided. The kits are useful for detecting and/or quantitating microorganisms and/or their gene expression (e.g., the expression of a toxin or a virulence gene). In one aspect, the kit can contain the probes and/or primers described in this paragraph. In one aspect, the primers or the probe can be fluorogenic (e.g., fluorescently labeled). In another embodiment, the kit can also contain components for nucleic acid amplification, such as a heat stable DNA polymerase (e.g., Taq polymerase or Vent polymerase), buffers, MgCl₂, H₂O, dNTPs, a reverse transcriptase, and the like. In one embodiment, the reagents can remain in liquid form. In another embodiment, the reagents can be lyophilized. In another illustrative embodiment, the kits can also contain instructions for use.

In one embodiment, a purified or isolated nucleic acid is provided comprising or consisting of a sequence of SEQ ID NO: 1 to SEQ ID NO: 15 or a sequence that hybridizes under highly stringent conditions to a sequence consisting of SEQ ID NO: 1 to SEQ ID NO: 15. In another embodiment, a purified or isolated nucleic acid is provided comprising a complement of a sequence of SEQ ID NO: 1 to SEQ ID NO: 15 or a sequence that hybridizes under highly stringent conditions to the complement of a sequence consisting of SEQ ID NO: 1 to SEQ ID NO: 15. In another embodiment, a kit comprising a purified or isolated nucleic acid with a sequence selected from the group of consisting of a sequence of SEQ ID NO: 6 or SEQ ID NO: 8 or a sequence that hybridizes under highly stringent conditions to a sequence consisting of SEQ ID NO: 6 or SEQ ID NO: 8. In another embodiment, a purified or isolated nucleic acid is also provided comprising a complement of a sequence of SEQ ID NO: 6 or SEQ ID NO: 8 or a sequence that hybridizes under highly stringent conditions to the complement of a sequence consisting of SEQ ID NO: 6 or SEQ ID NO: 8. In one embodiment, “highly stringent conditions” means hybridization at 65° C. in 5×SSPE and 50% formamide, and washing at 65° C. in 0.5×SSPE.

In another embodiment, the primer or probe, or a combination thereof, described herein is provided in a sterile container (e.g., a vial) or package, for example, an ampoule or a sealed vial.

As described herein the “card” can be, for example, an FTA® Card (Whatman® FTA® Card; for example, Whatman® catalogue numbers: WB12-0205, WB12-0206, WB12-0055, WB12-0056, WB12-0210, WB12-0210, WB12-0211, and WB12-0208; GE Healthcare Life Sciences, Pittsburgh, Pa.). Cards, such as a Whatman® FTA® Card, are conventionally used in the forensic sciences to collect, for example, blood or buccal cells. Whatman® FTA® Cards simplify the handling and processing of nucleic acids (e.g., DNA and RNA, including mRNA, an siRNA, an iRNA, a microRNA, etc.). Whatman® FTA® Cards contain chemicals that lyse cells, denature proteins, and protect nucleic acids from nucleases, oxidation and UV damage. Moreover, they rapidly inactivate organisms and prevent the growth of bacteria and other microorganisms. When a sample is applied to a Whatman® FTA® Card, cell membranes and organelles are lysed and the released nucleic acids are entrapped in the fibers of the matrix and are preserved (e.g., reduced degradation) throughout transport at room temperature. Upon arrival at a distant location, for example, the nucleic acid can be readily eluted from punches of the card through purification steps and prepared for downstream processing, as is known in the art. This technology also eliminates the labor intensiveness of selective plating and culture growth.

Moreover, this technology provides a start to finish process that encompasses all aspects of sample collection and analysis by utilizing cards, such as FTA® Cards. The cards also enable nucleic acid preservation in a sample from farm collection to long term lab storage and analysis. Stabilized nucleic acids can then be extracted from samples and tested for gene detection, quantification, and expression of a multitude of pathogenic microorganisms, such as bacteria. Complete sample analysis by the technology described herein constructs a broader view of pathogen-pathogen interaction, rather than singularly considering individual bacterial species effects. Thus, the present technology provides a more rapid, a more accurate, and a more cost effective analytical tool of identifying and understanding the greater pathogenic effects leading to total microbial load of agricultural species than is presently available.

The following examples provide illustrative methods for carrying out the practice of the present invention. As such, these examples are provided for illustrative purposes only and are not intended to be limiting.

### EXAMPLES

### Example 1

### Sample Collection and FTA Card Application

### Litter Samples

25 mL of the most sterile water available was added to a 50 mL conical tube. One full spoonful of litter material was added into the 50 mL conical tube and shaken vigorously for 30 seconds. Wood chips and other thick materials were allowed to briefly settle to the bottle. Using the transfer pipette, 125 μL of solution was added onto the FTA Card. Cards were allowed to dry in a cool dry area for 2-3 hours minimum. Card(s) were placed into a supplied zip bag with 2 desiccant packs.

### Swab Samples

Samples were collected by swabbing swine rectal or poultry cloaca to collect and absorb material. The swab was firmly pressed and rolled over the FTA Card application circle. Card(s) were allowed to dry in a cool dry area for 2-3 hours minimum. Card(s) were placed into a supplied zip bag with 2 desiccant packs.

### Example 2

### DNA Extraction

### Gram-Negative Bacteria

Six discs were punched (Miltex Biopsy Punch) from the card and placed in a 96 well block. 25 μL of Proteinase K and 180 μL of Buffer T1 were mixed for each sample. 200 μL of solution was added into each well of the Round-well Block. The Block was incubated at 56° C. for at least 6 hours (or optionally overnight). The Block was centrifuged to collect condensation. 200 μL of Buffer BQ1 and 200 μL of 96-100% ethanol were added to each sample. The samples were mixed vigorously by shaking for 10-15 seconds and briefly spun to collect the sample. Lysates were transferred into wells of a Tissue Binding Plate and spun at 5000 g for 10 min. 500 μL of Buffer BW was added and spun at 5000 g for 2 min. 700 μL of Buffer B5 was added, and spun at 5000 g for 4 min. The Binding Plate was placed onto an opened Rack of Tube Strips and incubated at 70° C. for 10 min to evaporate all the ethanol. The DNA was eluted by adding 100 μL of 70° C. preheated Buffer BE, and spun at 5000 g for 2 min.

### Gram-Positive Bacteria

Samples were pretreated with 180 μL Lysis Buffer and Lysozyme for at least 45 min at 37° C. The sample protocol was followed as stated for gram-negative bacteria. Concentrations were quantified using Quantus Fluorometer. dsDNA dye was prepared at a 1:200 concentration in 1× TE Buffer. 10 μL DNA, 90 μL 1× TE Buffer, and 100 μL of prepared dsDNA dye were added and vortexed. The tube was placed in a Fluorometer and measured.

### Example 3

### Quantitative Real-Time PCR

The amount of 2λ MasterMix, each primer and dH₂O was calculated based on reaction number, primer concentrations and reaction volume. Probe concentration can also be calculated if required for the reaction. All components were added, mixed, and distributed into PCR tubes. Samples were serially diluted at 10⁻¹ and the appropriate reference strain in 2 μL added to PCR tubes. 2 μL of Sample DNA template was added to each tube and the strips were vortexed. Each qPCR reaction was set up for the appropriate cycle conditions in accordance with the primer set used. Once the reaction was complete, the Bio-Rad program was used to analyze the Cq values in comparison to those of the reference strain.

### Example 4

### Example of DNA Quality from Litter Sample Collection

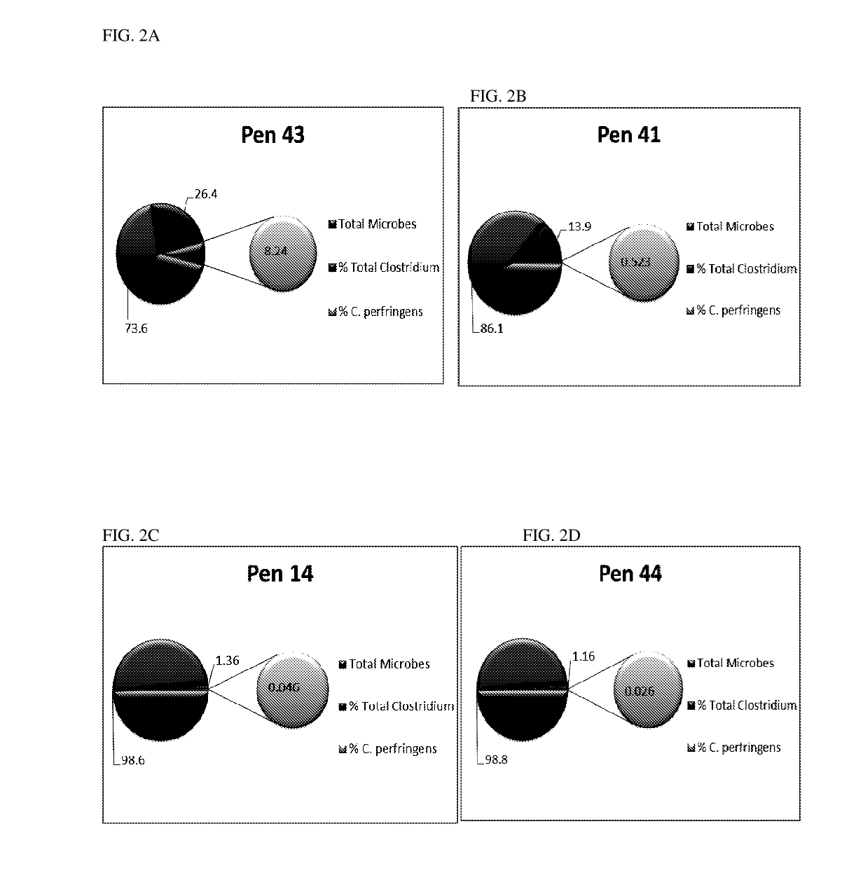

Measurements were performed on a Quantus Fluorometer Machine and results were as follows (see FIGS. 2A-2D):

Pen 14=0.402 ng/μL

Pen 41=0.418 ng/μL

Pen 43=0.502 ng/μL

Pen 44=0.299 ng/μL

DNA Normalization Calculations

V₁C₁=V₂C₂ Equation:

### Example 5

### qPCR Reactions

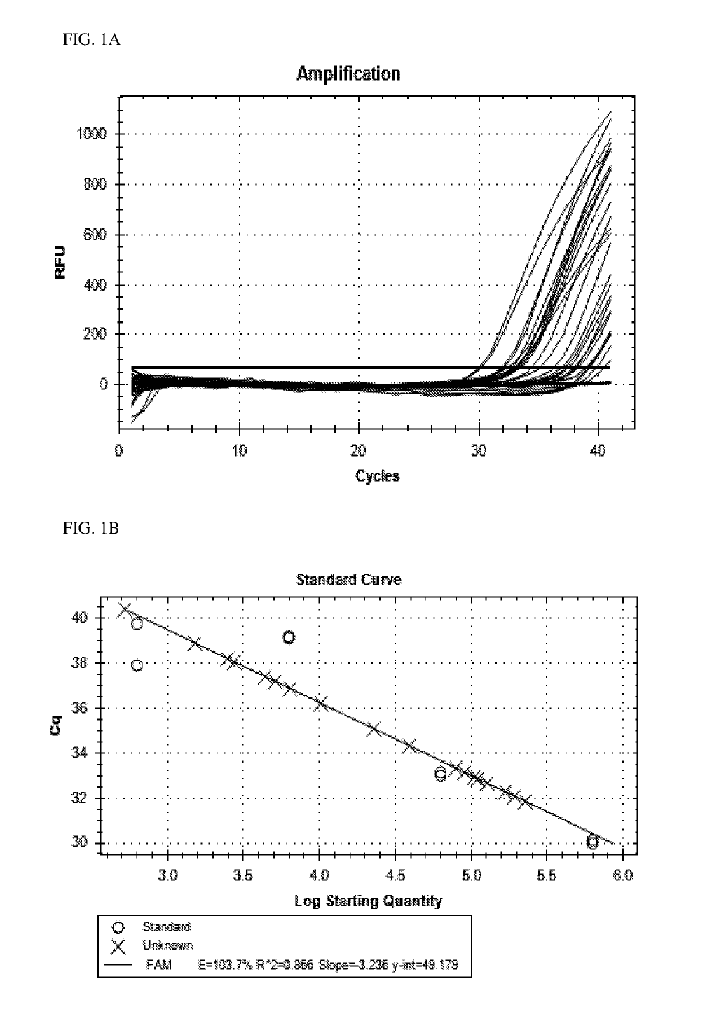

Quantitative PCR amplifies purified DNA based on specifically designed primers which target a particular region in the gene sequence. In addition, qPCR goes one step further by incorporating a fluorogenic probe to enable real-time measurements of fluorescence as the DNA is amplified to quantify the sample rather than determining this based on band intensity in end point PCR. The oligonucleotide probe also adds a heightened specificity factor. The probes are designed specifically to target a gene sequence and fluoresce only when bound, therefore the Thermocycler measures when the probe is bound specifically to the target gene dsDNA whereas end point PCR amplifies any dsDNA. Amplification was measured as a Cq Value (FIG. 1A) based on how many cycles it takes the DNA sample to begin amplifying and the strength of the fluorescence was measured in RFU values. The lower the Cq value, the less cycles it took to amplify, therefore the more target gene was present. Quantification numbers were derived from a Standard Curve (FIG. 1B) which was calculated from a serially diluted reference strain with a known starting initial count. Acceptable standard curves achieved a slope between −3.1 and −3.6 giving reaction efficiencies between 80 and 110%.

### Example 6

### Relative Quantity Calculations

Relative quantities were calculated as a percentage of the total microbial load from each of the Cq Values. qPCR reactions were run at the species level (Clostridium perfringens), the genus level (Clostridium spp.), and for total microbes. As described herein, the Clostridium counts were comprised from Clostridium Clusters I, IV, and XIV which encompass the majority of the intestinal Clostridium. FIGS. 2A, 2B, 2C, and 2D show examples of poultry litter samples taken from a farm trial. The pie charts represent the amount of C. perfringens within the total Clostridium spp. within the total microbial load.

### Example 7

### Absolute Quantity Calculations

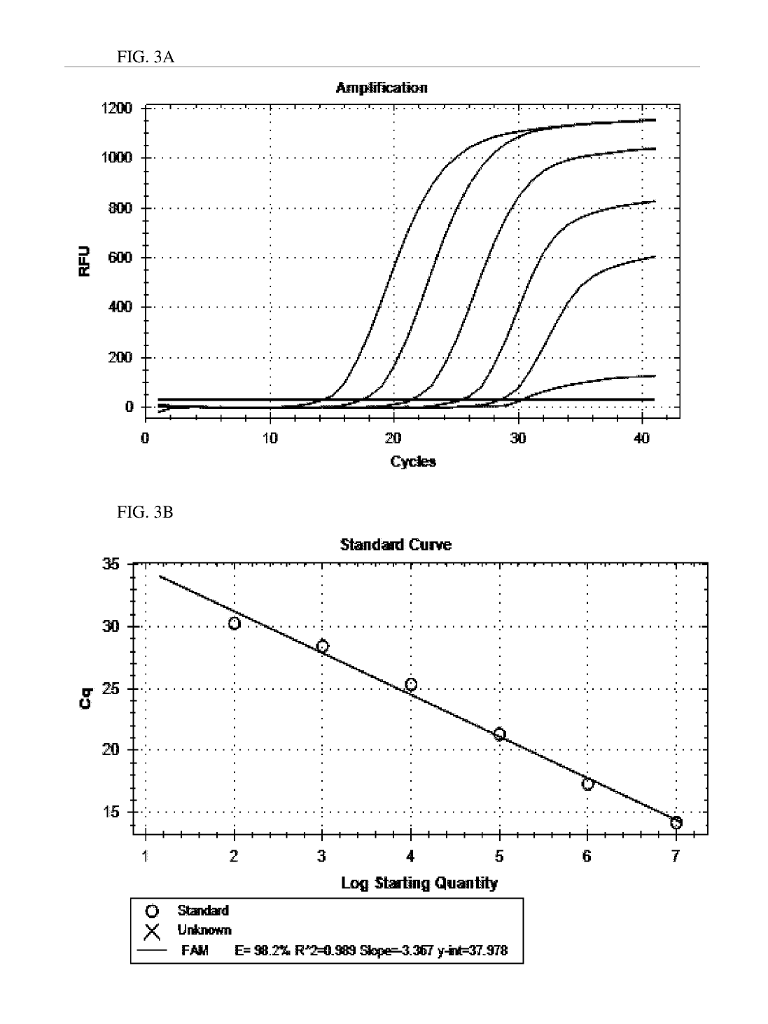

Absolute quantities were calculated from a standard curve created from a serially diluted reference strain with a known initial count (See FIG. 3A). This technology is used to quantify the absolute amount of each target gene in a sample. Starting quantities of a sample were calculated by determining where its Cq value falls on the linear curve (See FIG. 3B). Counts are reported as gene copies rather than exact counts due to target genes having variable gene copies per cell.

### Example 8

### Quantification of Clostridium perfringens

As previously described, absolute quantities were derived from a standard curve created from a serially diluted reference strain with a known initial count. This technology was used to quantify the absolute amount of C. perfringens in a sample. SQ values represent the calculated count for each sample.

TABLE 2
Quantification of Clostridium perfringens.

| Fluor | Target | Sample | Cg | SQ | Fluor | Target | Sample | Cg | SQ |
|---|---|---|---|---|---|---|---|---|---|
| FAM | | Neg Ctrl | N/A | N/A | FAM | C. perf | P13-T1 | 33.33 | 7.91E+04 |
| FAM | | Neg Ctrl | N/A | N/A | FAM | C. perf | P43-T1 | 32.27 | 1.68E+05 |
| FAM | C. perf | Std-1 | 29.97 | 6.30E+05 | FAM | C. perf | P43-T1 | 32.66 | 1.27E+05 |
| FAM | C. perf | Std-1 | 30.13 | 6.30E+05 | FAM | C. perf | P2-T2 | 31.85 | 2.27E+05 |
| FAM | C. perf | Std-2 | 33.14 | 6.30E+04 | FAM | C. perf | P2-T2 | 32.07 | 1.93E+05 |
| FAM | C. perf | Std-2 | 32.99 | 6.30E+04 | FAM | C. perf | P14-T2 | 34.32 | 3.91E+04 |
| FAM | C. perf | Std-3 | 39.17 | 6.30E+03 | FAM | C. perf | P14-T2 | 35..07 | 2.29E+04 |
| FAM | C. perf | Std-3 | 39.1 | 6.30E+03 | FAM | C. perf | P44-T2 | 32.93 | 1.05E+05 |
| FAM | C. perf | Std-4 | 39.73 | 6.30E+02 | FAM | C. perf | P44-T2 | 32.86 | 1.11E+05 |
| FAM | C. perf | Std-4 | 37.89 | 6.30E+02 | FAM | C. perf | P6-T3 | 38.19 | 2.49E+03 |
| FAM | C. perf | Std-5 | N/A | 6.30E+01 | FAM | C. perf | P6-T3 | 38.89 | 1.51E+03 |
| FAM | C. perf | Std-5 | N/A | 6.30E+01 | FAM | C. perf | P17-T3 | 36.86 | 6.41E+03 |
| FAM | C. perf | P1-T1 | 37.17 | 5.13E+03 | FAM | C. perf | P17-T3 | 37.39 | 4.41E+03 |
| FAM | C. perf | P1-T1 | 36.2 | 1.02E+04 | FAM | C. perf | P41-T3 | 38.05 | 2.76E+03 |
| FAM | C. perf | P13-T1 | 33.14 | 9.04E+04 | FAM | C. perf | P41-T3 | 40.38 | 5.24E+02 |

### Example 9

### Toxin Detection

Methods described herein were used to determine the presence or absence along with either absolute or relative quantities of specific toxin associated genes for a pathogenic bacterial species of interest. An important distinction in this technique is detection of the presence or absence of the gene from a DNA sample and not measuring the amount of toxin gene expressed. Having knowledge of which toxin genes are in the samples is important in assessing the risk for diseases. RNA from the samples was accessed to determine how much of the toxin was actually being produced and expressed, as that directly correlates to disease occurrence. Toxin detection was also achieved through qPCR reactions that measured amplification of sample DNA in relation to a known reference strain containing the target gene.

Another aspect of this technology is the ability to determine quantities of specific toxin or virulence associated genes for pathogenic bacteria of interest. Absolute quantities of any plasmid-borne toxin gene may not be able to be determined since they are capable of horizontal gene transfer resulting in an unknown number of copies per DNA sample, and thus reported as copy number. However, toxin genes that are chromosomally-borne can be quantified because there will be one chromosome per DNA amplified, which is reported as colony-forming unit or CFU.

There were approximately 37 genes validated for toxin detection according to the following: 1) BV4-5 region for universal bacteria, 2) 16s gene of Clostridium Cluster I, 3) 16s gene of Clostridium Cluster IV, 4) 16s gene of Clostridium Cluster XIV, 5) 16s gene of Clostridium perfringens, 6) cpn60 gene of Clostridium perfringens, 7) cpa toxin gene of Clostridium perfringens, 8) cpb toxin gene of Clostridium perfringens, 9) cpb2 toxin gene of Clostridium perfringens, 10) cpe toxin gene of Clostridium perfringens, 11) etx toxin gene of Clostridium perfringens, 12) 16s gene of Clostridium difficile, 13) tcdA toxin gene of Clostridium difficile, 14) tcdB toxin gene of Clostridium difficile, 15) 16s gene of E. coli, 16) Stx1 toxin gene of E. coli, 17) Stx2 toxin gene of E. coli, 18) LT toxin gene of E. coli, 19) STa toxin gene of E. coli, 20) STb toxin gene of E. coli, 21) eaeA virulence factor gene of E. coli, 22) EAST1 toxin gene of E. coli, 23) hlyF virulence factor gene of E. coli, 24) ompT virulence factor gene of E. coli, 25) iroN virulence factor gene of E. coli, 26) iutA virulence factor gene of E. coli, 27) iss virulence factor gene of E. coli, 28) 16s gene of Campylobacter spp., 29) cpn60 gene of Campylobacter jejuni, 30) CDT toxin of Campylobacter jejuni, 31) cpn60 gene of Campylobacter coli, 32) invA gene of Salmonella spp., 33) fliC virulence factor gene of Salmonella enterica enterica Typhimurium, 34) sefA virulence factor gene of Salmonella enterica enterica Entertidis, 35) cpsJ2 virulence gene of Streptococcus suis, 36) P46, P97, and P107 virulence proteins of Mycoplasma hyopneumoniae, and 37) Omp virulence gene of Haemophilus parasuis.

### Example 10

### Toxin Expression

In addition to toxin and virulence gene quantification, this technology also determines the amount of a gene that is present that is actually expressed. Gene presence determines the potential of the gene. However, the expression level of a gene more accurately represents a risk of that gene for pathogenesis. Thus, gene expression is accomplished by extracting RNA rather than DNA, reverse transcribing the RNA product into cDNA, and then analyzing the resulting cDNA in a real-time PCR reaction. Similar to gene detection analysis, gene expression analysis requires gene-specific primers designed in a particular gene region to amplify a target sequence.

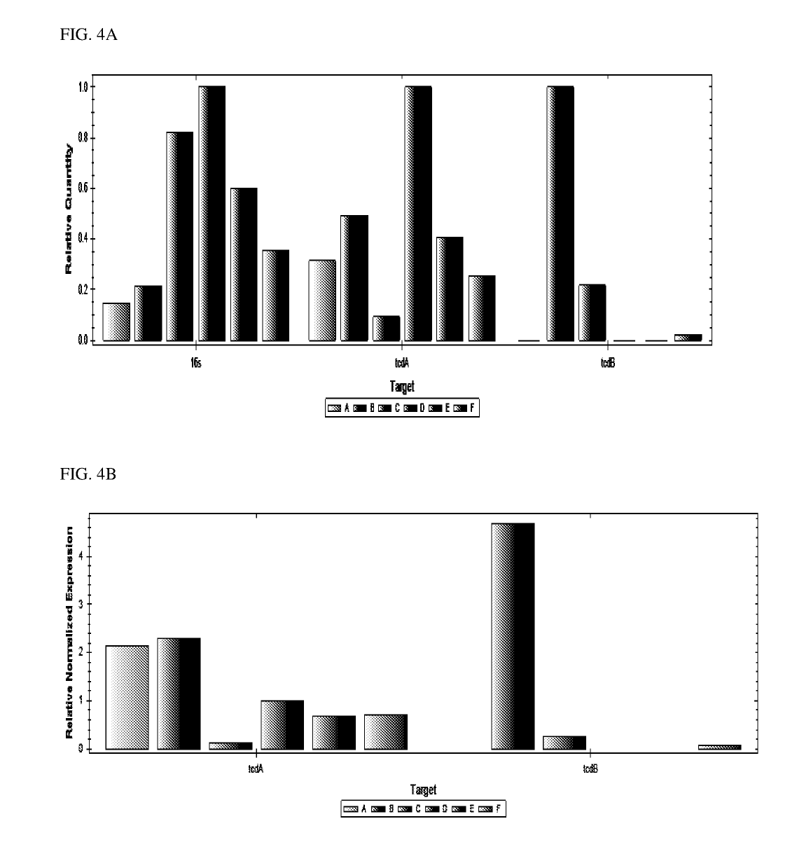

Gene expression analysis requires a validated and constitutively expressed housekeeping gene to be used as a reference gene. Relative quantity levels of the gene of interest are then compared to the relative quantity of the corresponding reference gene in order to determine relative normalized expression levels. FIG. 4A illustrates the relative quantity levels of the 16S reference gene and the tcdA and tcdB toxin genes of interest. FIG. 4B illustrates the normalized expression levels of the tcdA and tcdB toxin genes after being normalized by the 16S reference gene expression level. This technology enables a better understanding as to which toxin and/or virulence genes may be contributing to symptoms of pathogenic diseases.

There were approximately 18 validated genes for toxin expression according to the following: 1) 16s (Clostridium perfringens reference gene), 2) rpoA (Clostridium perfringens single copy reference gene), 3) cpa toxin gene of Clostridium perfringens, 4) cpb toxin gene of Clostridium perfringens, 5) cpb2 toxin gene of Clostridium perfringens, 6) cpe toxin gene of Clostridium perfringens, 7) etx toxin gene of Clostridium perfringens, 8) 16s (Clostridium difficile reference gene), 9) tcdA toxin gene of Clostridium difficile, 10) tcdB toxin gene of Clostridium difficile, 11) GAPDH (E. coli reference gene), 12) Stx1 toxin gene of E. coli, 13) Stx2 toxin gene of E. coli, 14) LT toxin gene of E. coli, 15) STa toxin gene of E. coli, 16) STb toxin gene of E. coli, 17) eaeA virulence factor gene of E. coli, and 18) EAST1 toxin gene of E. coli.

### Example 11

### qPCR Analysis of FTA Cards (16S rDNA Trial)

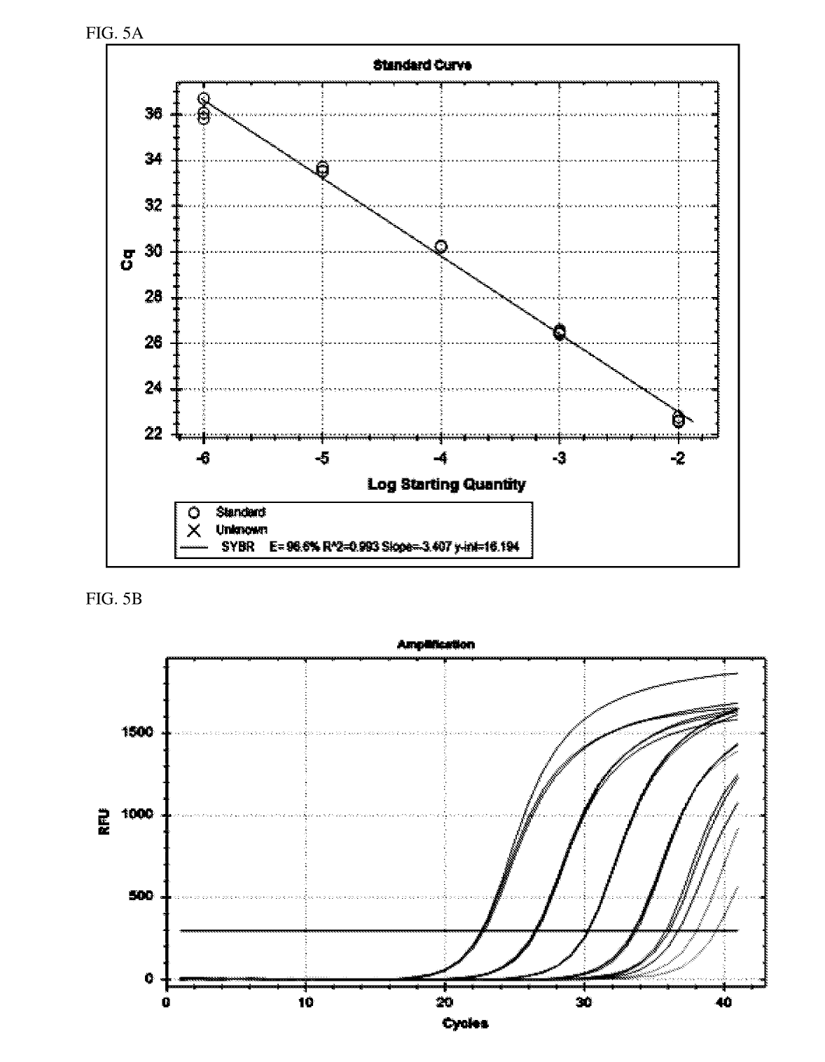

The performance of a universal bacterial 16S rDNA qPCR assay with DNA from cells in pond water preserved on FTA cards was analyzed. Control DNA for assay validation consisted of serial dilutions of Vibrio campbellii genomic DNA in sterile water, run in triplicate. V. campbellii-spiked FTA cards from a previous Vibrio detection study with concentrations between 5.8×10⁸ CFU/ml and 5.8×10³CFU/ml were used as quantification standards for the card method. FTA cards from a shrimp farm were the unknowns. All samples were run in triplicate. Quantitative PCR was performed using a 20 μl reaction mixture of Bio-iTaq SYBR Green Supermix (1×), universal bacterial 16S primers 1099F and 1510R from (Reysenbach et al., Appl Environ Microbiol. 1994 June; 60(6): 2113-2119)(400 nM each), and 5 μl of template DNA extracted from FTA cards. No-template controls were included. Cycling conditions were designed with the protocol auto-writer in Bio-Rad's CFX Manager software and were as follows: 3 minutes at 95° C., 40 cycles of (10 seconds at 95° C., 20 seconds at 55° C. 20 seconds at 72° C. followed by a plate read), followed by melt curve analysis from 65° C. to 95° C. in 0.5° C. increments. Results using genomic DNA controls (E=96/6%, R²=0.993) are shown in FIG. 3, Panels A and B. Amplification after 33 cycles occurred in no template controls, but this is common with universal primers due to E. coli gDNA contamination in most Taq polymerases and other PCR enzymes (See FIGS. 5A and 5B).

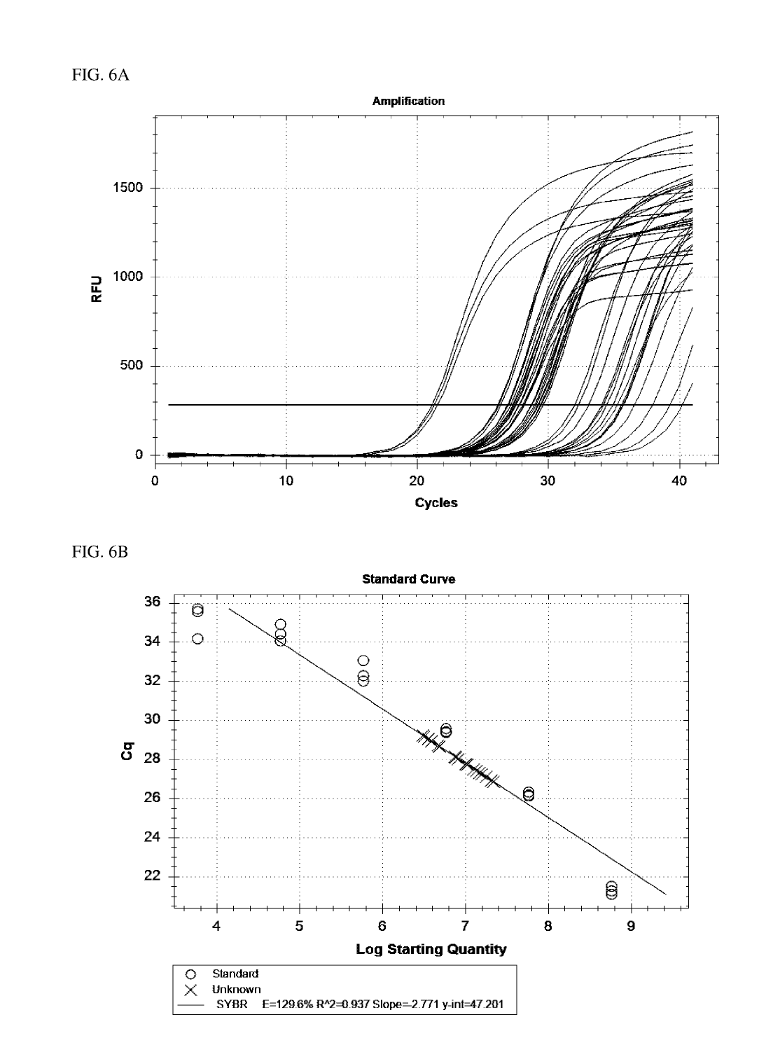

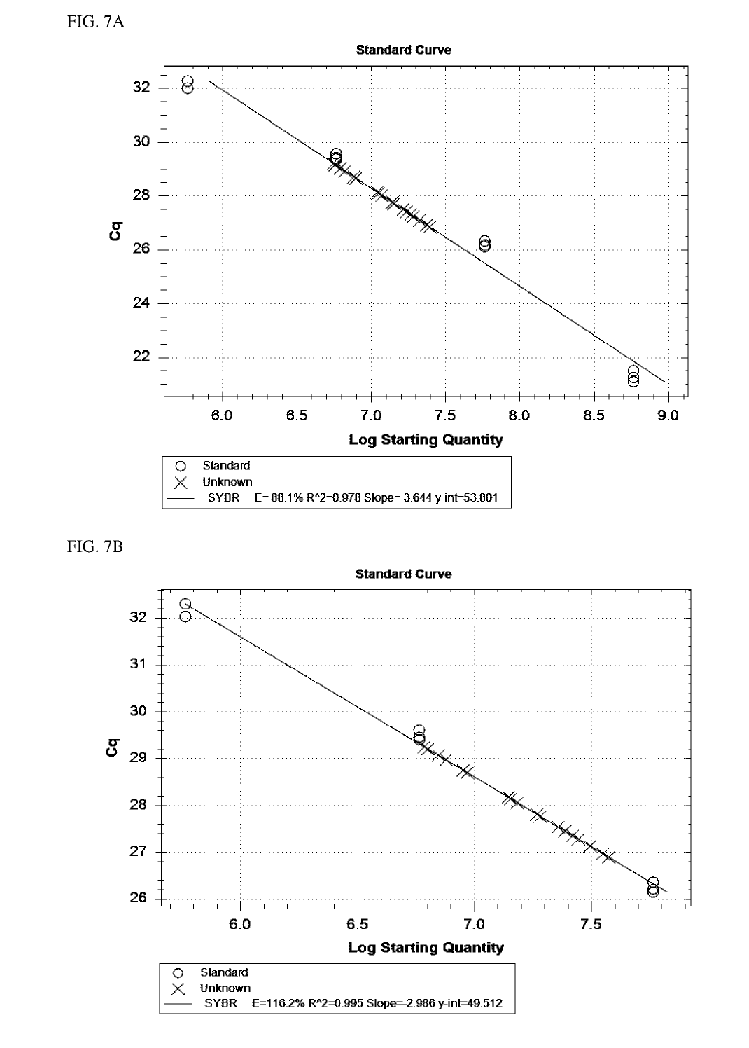

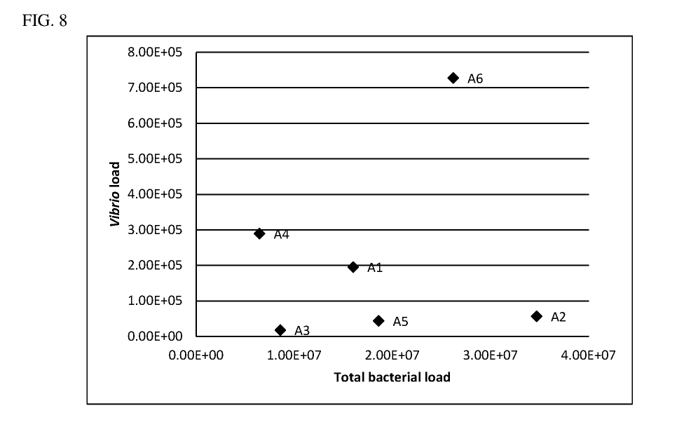

The standard curves for FTA card controls and samples are shown in FIGS. 6A and 6B. Quantification data varied depending on the points used to construct a standard curve. The most conservative version excludes the 5.8×10³ CFU/ml and 5.8×10⁴ CFU/ml data points, as they overlap with no template controls, and one outlier point, keeping a 4-log range on the standard curve (See FIG. 7A). Eliminating the 5.8×10⁸ CFU/ml dilution improved the fit of the standard curve but reduced the range (See FIG. 7B). Correlation between total bacterial load and total Vibrio load in the samples tested is shown in FIG. 8.

TABLE 3
Quantification data varied depending on the version of the curve used

| 3 log curve | | | | 4 log curve | | | |
|---|---|---|---|---|---|---|---|
| | Mean | | | | Mean | | |
| | starting | Standard | | | starting | Standard | |
| Pond | quantity | deviation | 95% Confidence Interval | Pond | quantity | deviation | 95% Confidence Interval |
| A1 | 1.60E+07 | 2.19E+06 | 1.38E+07 to 1.82E+07 | A1 | 1.23E+07 | 1.40E+06 | 1.09E+07 to 1.37E+07 |
| A2 | 3.47E+07 | 3.18E+06 | 3.15E+07 to 3.79E+07 | A2 | 2.32E+07 | 1.82E+06 | 2.14E+07 to 2.50E+07 |
| A3 | 8.59E+06 | 9.34E+05 | 7.66E+06 to 9.52E+06 | A3 | 7.39E+06 | 6.50E+05 | 6.74E+06 to 8.04E+06 |
| A4 | 6.46E+06 | 4.72E+05 | 5.99E+06 to 6.93E+06 | A4 | 5.83E+06 | 3.42E+05 | 5.49E+06 to 6.17E+06 |
| A5 | 1.86E+07 | 4.44E+06 | 1.42E+07 to 2.30E+07 | A5 | 1.39E+07 | 2.74E+06 | 1.12E+07 to 1.66E+07 |
| A6 | 2.62E+07 | 1.74E+06 | 2.45E+07 to 2.79E+07 | A6 | 1.83E+07 | 9.79E+05 | 1.73E+07 to 1.93E+07 |

TABLE 4
Correlation between total bacterial load and total Vibrio
load in the samples

| | Total quantity (Universal | |
|---|---|---|
| Sample | 16S) | Vibrio Quantity (Vibrio 16S) |
| A1 | 1.60E+07 | 1.95E+05 |
| A2 | 3.47E+07 | 5.63E+04 |
| A3 | 8.59E+06 | 1.77E+04 |
| A4 | 6.46E+06 | 2.89E+05 |
| A5 | 1.86E+07 | 4.33E+04 |
| A6 | 2.62E+07 | 7.27E+05 |

### Example 12

### Detection of the V. harveyi and V. campbellii Hemolysin Gene in FTA Card Samples

Vibrio harveyi and Vibrio campbellii were detected in water samples and FTA card samples via PCR assays targeting their hemolysin (hly) gene sequences, and the expression of the hemolysin gene in FTA card samples was analyzed. An endpoint PCR assay was used to detect presence or absence of the hly gene in diluted pond water samples and pond water samples stored dry on FTA cards. The PCR assay was evaluated for performance in quantitative PCR with water samples and FTA card samples. A reverse primer was developed to amplify a smaller section of the hly gene than the original assay (for better performance in qPCR) and checked for specificity against published V. harveyi and V. campbellii sequences. Additional PCR assays for total Vibrio and total bacteria were used to determine the proportional abundance of V. campbellii and V. harveyi in pond water samples. The stability of V. harveyi RNA on FTA cards was evaluated to determine the possibility of gene expression analysis. The PCR assay was evaluated for performance in qRT-PCR gene expression analysis, detecting hly mRNA in liquid culture. The sensitivity of the qRT-PCR assay was evaluated using V. harveyi RNA extracted from FTA cards.

### FTA Card Sampling and Storage:

Performed as directed by the manufacturer.

### FTA Card DNA Extraction:

For endpoint PCR, the manufacturer's directions for amplification directly from an FTA card sample punch are used. For quantitative PCR, DNA was eluted from the cards with the Qiagen DNeasy Mini Kit. (Protocol: DNA Purification from Dried Blood Spots. A nearly identical procedure with the QiaAmp DNA investigator Kit is listed in GE Life Science's application note 28-9822-22 AA).

### FTA Card RNA Extraction:

The manufacturer's protocol was used for extraction with an RNA processing buffer (Preparation of RNA from Blood and Tissue Culture on FTA® Cards for RT-PCR or Northern Blot Analysis) or the Qiagen RNeasy Mini kit.

V. campbellii and V. harveyi Hly PCR Assays:

Primer sets are as follows:

TABLE 5
Vibrio PCR primers and assays
PCR primers and assays

| Target | Forward primer | Reverse primer | qPCR cycle |
|---|---|---|---|
| Vibrio | CTATTGGTGGAACGCAC | GTATTCTGTCCATACAAAC | 3 min @ 95° C., 40 |
| campbellii | (SEQ ID NO. 1) | (SEQ ID NO. 2) | cycles of (10 s @ |
| hly | | | 95° C., 20 s @ |
| | | | 54° C., 20 s @ |
| | | | 72° C.) |
| | | | 3 min @ 95° C., 40 |
| Vibrio | GAGTTCGGTTTCTTTCAA | TGTAGTTTTTCGCTAATTTC | cycles of (10 s @ |
| harveyi | (SEQ ID NO. 3) | (SEQ ID NO. 4) | 95° C., 20 s @ |
| hly | | | 54° C., 20 s @ |
| | | | 72° C.) |
| Vibrio | CTATTGGTGGAACGCAC | CAGCGAAGTAGGTAATGTC | 3 min @ 95° C., 40 |
| campbellii | (SEQ ID NO. 5) | (SEQ ID NO. 6) | cycles of (10s @ |
| hly (short | | | 95° C., 30 s @ |
| amplicon) | | | 55° C.) |
| Vibrio | GAGTTCGGTTTCTTTCAA | AAACGGTTATCGGCTG | 3 min @ 95° C., 40 |
| harveyi | G | (SEQ ID NO. 8) | cycles of (10s @ |
| hly (short | (SEQ ID NO. 7) | | 95° C., 30 s @ |
| amplicon) | | | 55° C.) |
| Vibrio | GGCGTAAAGCGCATGCA | GAAATTCTACCCCCCTCTACA | 3 min @ 95° C., 40 |
| 16S rRNA | GGT (SEQ ID NO. 9) | G (SEQ ID NO. 10) | cycles of (10s @ |
| | | | 95° C., 30s @ |
| | | | 55° C.) |
| Bacterial | 27F: | 1492R: | 3 min @ 95° C., 40 |
| 16S rRNA | AGAGTTTGATCMTGGCTC | GGTTACCTTGTTACGACTT | cycles of (10s @ |
| | AG (SEQ ID NO. 11) | (SEQ ID NO. 12) | 95° C., 30 s @ |
| | | | 55° C.) |

### Example 13

### RNA Extraction and qRT-PCR

RNA was extracted from 4×2.0 mm punches for determination of yield, or half the sampling area of each FTA card for subsequent gene expression analysis, with the RNeasy Mini kit (Qiagen) with on-column DNase digestion and an additional DNase digestion in solution, followed by RNA cleanup with RNeasy mini. RNA was quantified with the fluorometric method and 80 ng of RNA from each treatment was reverse transcribed with iScript Reverse transcriptase (Bio-Rad) in duplicate, and SYBR Green quantitative PCR (Bio-Rad iTaq SYBR Green Supermix) was performed using 2 μl of cDNA template per 20 μl reaction, 3 technical replicates per RT reaction. Genes amplified were hly and Vibrio-specific 16S rRNA. Relative normalized expression with PCR efficiency correction was computed via the ΔΔC_{q} method in CFX Manager.

RNA yield from 2-day-old FTA cards ranged from <5 ng (with the Direct-Zol kit) to 610 ng (with the Whatman RNA processing buffer). The low-yielding Direct-Zol method was excluded from further analysis. The effect of storage temperature was determined with samples extracted with the high-yield method (Whatman's RNA processing buffer). RNA loss was not observed after 20 days of storage at −20 or 20° C. A 25% decrease was observed at 37° C., but yield remained above 400 ng per 5-punch extraction, an amount sufficient for reverse transcription with standard kits such as the iScript RT supermix used in this study.

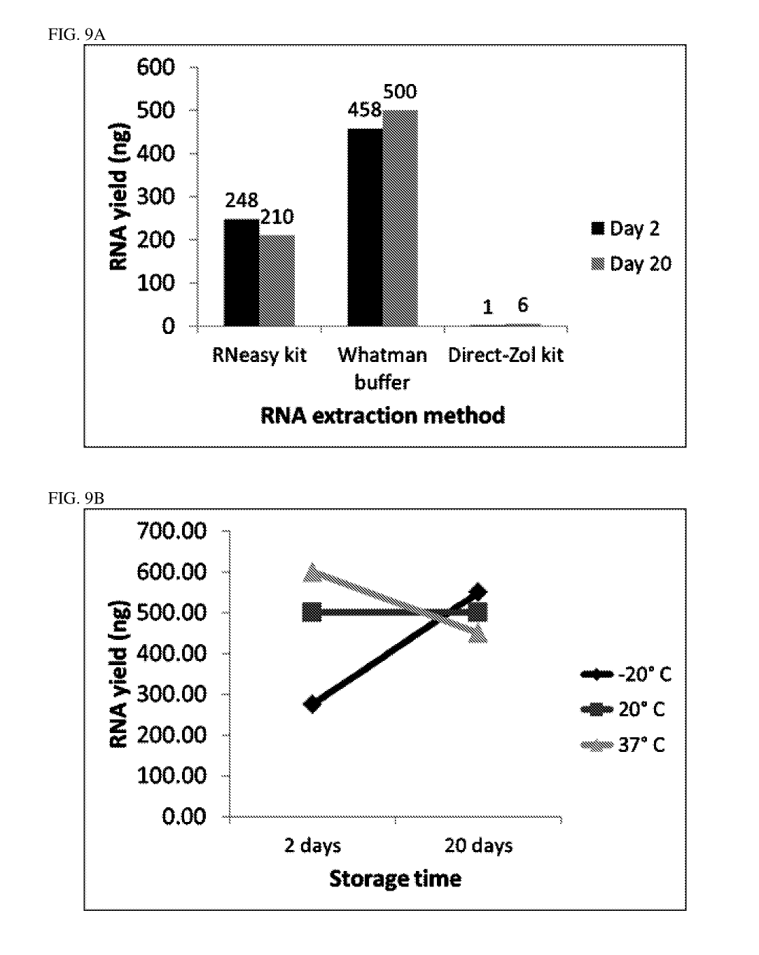

RNA was successfully recovered from FTA cards stored for at least 2 months, with yields from pure culture stored on FTA cards as high as 500 ng per 5-punch prep, or 100 ng per 4 mm punch. No decline in RNA concentration was detected in the room-temperature or frozen samples over the course of the experiment. A 25% decline in RNA yield was observed at 37° C. with sustained storage, but FTA cards will still be suitable for shipment from remote locations where short periods of thermal stress during shipping are expected (See FIGS. 9A and 9B).

### Example 14

### Aquaculture Pond Analysis

Pond water samples on FTA cards collected from six shrimp ponds in Vietnam were assayed for V. campbellii, V. harveyi, total Vibrio, and total bacteria with the qPCR assays described above, using 18×2.0 mm FTA card discs per DNA extraction and 100 μl of template DNA per qPCR. The standard curve for quantification consisted of serial tenfold dilutions of Vibrio harveyi and V. campbellii cells applied to FTA cards and extracted with the same method.

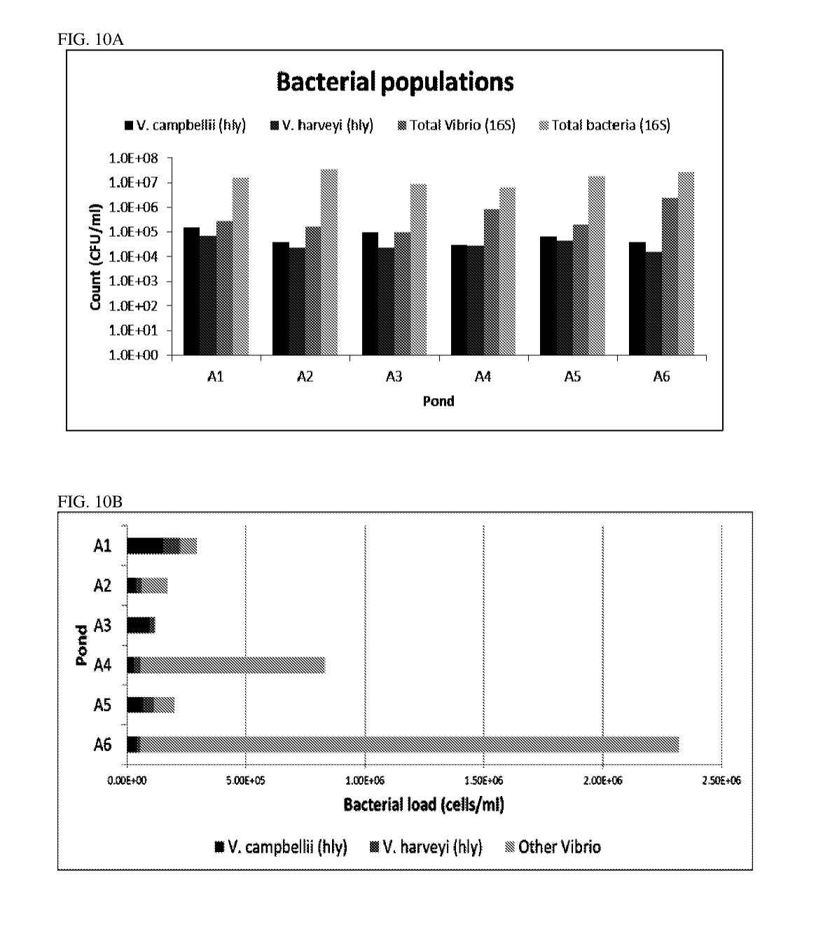

V. harveyi and V. campbellii concentrations ranged from 1.5×10⁴ to 1.5×10⁵ cells/ml in the pond samples tested, while total Vibrio concentration ranged from 9.7×10⁴ to 2.3×10⁶ cells per ml and estimated total bacterial population ranged from 6.5×10⁶ to 3.5×10⁷ cells/ml. In all ponds tested, V. campbellii and V. harveyi represented less than 2% of estimated bacterial count. In ponds A4 and A6, other Vibrio were dominant, representing 8-11% of estimated bacterial abundance (See FIGS. 10A and 10B).

### Example 15

### Alternative RNA Extraction and qRT-PCR Method

RNA was extracted from 12×2.0 mm punches for determination of yield, and placed in a 1.5 ml centrifuge tube. Two times the volume of RNAprotect Bacterial Reagent was placed in the centrifuge tube, and 1) incubated for 5 min at RT, 2) centrifuged for 10 min at 8000 rom, and 3) the supernatant was decanted. 200 μl of Proteinase K was added to 200 μl of TE buffer containing lysozyme (at 20 mg/mL) and added to the tube. The mixture was incubated at RT for 45 mins with continuous shaking. 700 μl of Buffer RLT was added and the mixture was vortexed vigorously. 500 μl of 96%-100% ethanol was added to the tube and mixed using a pipet. 700 μl of the lysate was transferred to a RNeasy Mini Spin column in a 2 mL collection tube, centrifuged at 8000 rpm for 15 sec, and repeated. 350 μl of Buffer RW1 was added and centrifuged at 8000 rpm for 15 sec. Separately, 10 μl of DNAse I was added to 70 μl Buffer RDD and mixed by inversion. 80 μl of that solution was directly added to a column membrane and incubated for 15 min at RT. 500 μl of Buffer RPE was added and centrifuged at 8000 rpm for 15 sec. An additional 500 μl of Buffer RPE was added and centrifuged at 8000 rpm for 2 min. Optionally, the column may be centrifuged for an additional 1 min to prevent ethanol carryover. After centrifuging, the column was placed in a new 1.5 mL tube, 600 μl of RNase-free water was added and centrifuged for 1 min at 8000 rpm. Alternatively, 30 μl of RNase-free water may be added and centrifuged to increase RNA concentration.

For each RNA sample, 16 μl of eluted RNA was added to two different tubes (i.e., Tube 1 and Tube 2). 4 μl of Reverse Transcriptase Supermix was added to Tube 1. 4 μl of No-RT Supermix Control was added to Tube 2. Each tube underwent a PCR reaction with the following conditions: 25° C. for 5 mins, 42° C. for 30 mins, and 85° C. for 5 mins.

Real-Time qPCR reactions were prepared by calculating the amount of 2× MasterMix, each primer, and dH₂O based on the reaction number, primer concentration, and reaction volume. All components were added and mixed in a PCR tube. 2 μl of RNA template was added to each tube and vortex strips. The qPCR reactions, including reference genes and samples of interested, were set up for the appropriate cycle conditions in accordance with the primer set used. Once the qPCR reactions were complete, the Bio-Rad program was used to analyze the Cq values, and to determine the relative difference in quantity and expression between the reference gene (baseline control) and the sample of interest.

### Example 16

### Detection of Campylobacter Spp. and Clostridium Spp. Genes in FTA Card Samples

The previously used elution method (for vibrio) was not optimal for Clostridium because the gram+ structure of Clostridium is tougher to lyse. For Clostridium spp., DNA isolation protocols were simultaneously performed incorporating different aspects from the Dried Blood Spot Protocol (for vibrio) and the gram+ bacteria pretreatment protocol. Lysozyme pretreatment and the addition of proteinase K and an AL lysis buffer were added to the samples using varying combinations of temperatures and times to determine which yielded the greatest DNA concentration and lowest Cq values. The gram+ bacteria pretreatment protocol was followed through the additional of ethanol step and then the spin column process was finalized from the dried blood spot protocol yielding the best DNA results.

Primer Sets:

1) 16S—Universal Bacteria BV4-5; Clostridium Clusters I, IV, and XIV; Campy spp.

2) Cpn60—C. perfringens; C. jejuni; C. coli

3) Toxin Gene—cpe and cpb

4) Universal Bacteria (cpn60)

5) Clostridium Family Clusters—I, IV, XIV

Results will show, for example, relative quantification of total clostridium in relation to universal bacteria, absolute quantification of C. perf, cpe, and cpb toxin genes, and absolute quantification of Campy spp., C. jejuni and C. coli.

TABLE 6
Primer Set Conditions

| | Rxn Type | Target | Primer | Probe | Anneal | | Conditions |
|---|---|---|---|---|---|---|---|
| Universal | SYBR | 16 s | 200 nM | X | 60 | C. | 95 C. 3 m, (95 C. 10 s + 60 C. |
| | | | | | | | 30 s + 72 C. 1 m) × 39 |
| CC I | SYBR | 16 s | 200 nM | X | 57.5 | C. | 95 C. 3 m, (95 C. 10 s + 57.5 C. |
| | | | | | | | 30 s + 72 C. 1 m) × 39 |
| CC IV | SYBR | 16 s | 200 nM | X | 57.5 | C. | 95 C. 3 m, (95 C. 10 s + 57.5 C. |
| | | | | | | | 30 s + 72 C. 1 m) × 39 |
| CC XIV | SYBR | 16 s | 200 nM | X | 60 | C. | 95 C. 3 m, (95 C. 10 s + 60 C. |
| | | | | | | | 30 s + 72 C. 1 m) × 39 |
| C. perf | TaqMan | Cpn60 | 500 nM | 200 nM | 59 | C. | 94 C. 2 m, (94 C. 30 s + 59 C. |
| | | | | | | | 30 s) × 40 |
| Cpe/cpb | TaqMan | Gene | 250 nM | 100 nM | 54 | C. | 95 C. 30 s, (95 C. 30 s + 54 C. |
| | | | | | | | 1 m) × 40 |
| Campy spp. | SYBR | 16 s | 1000 nM | X | 61 | C. | 95 C. 5 m,(95 C. 15 s + 61 C. |
| | | | | | | | 20 s + 72 C. 45 s) × 39 + 72 C. 5 m |
| C. jejuni | SYBR | Cpn60 | 400 nM | X | 62.5 | C. | 95 C. 3 m, (95 C. 15 s + 62.5 C. |
| | | | | | | | 15 s + 72 C. 15 s) × 40 + 95 C. 1 m |
| C. coli | SYBR | Cpn60 | 400 nM | X | 64.6 | C. | 95 C. 3 m, (95 C. 15 s + 64.6 C. |
| | | | | | | | 15 s + 72 C. 15 s) × 40 + 95 C. 1 m |

TABLE 7
Testing of Samples (*Pen 41- Positive for cpb toxin gene)

| | Universal | CC I/% | CCIV/% | CCXIV/% | % of Uni | C. perf |
|---|---|---|---|---|---|---|
| SWABS | | | | | | |
| Pen 13-C | 17.84 | 22.91/4.6% | 35.76/.002% | 28.45/0.16% | 4.76% | 3.28e6 |
| Pen 43-C | 16.63 | 23.56/1.5% | 24.30/1.0% | 18.99/23.9% | 26.4% | 1.50e6 |
| Pen 2-N | 18.58 | 35.26/.004% | 27.02/0.60% | 23.25/5.90% | 6.51% | 2.71e3 |
| Pen 14-N | 21.47 | 38.43/.003% | 33.71/0.06% | 28.69/1.30% | 1.36% | 2.18e2 |
| Pen 44-N | 17.96 | 36.16/.002% | 30.22/0.06% | 25.44/1.10% | 1.16% | 4.31e3 |
| Pen 6-B | 22.86 | 39.79/.004% | | | | 5.35e2 |
| Pen 17-B | 17.17 | 37.59/.0004% | 30.64/0.03% | 24.54/1.10% | 1.13% | X |
| Pen 41-B | 19.17 | 31.78/.05% | 27.84/0.52% | 22.49/13.4% | 13.9% | 2.00e4* |
| GS- ILEUM | | | | | | |
| Pen 1-C | 22.21 | X | X | X | | X |
| Pen 13-C | 19.84 | 27.94/.55% | X | 32.02/0.04% | 0.59% | 3.33e5 |
| Pen 2-N | 21.58 | 34.74/.02% | X | 32.56/0.09% | 0.11% | 6.37e2 |
| Pen 14-N | 21.85 | 38.87/.002% | X | 31.50/0.20% | 0.20% | X |
| Pen 6-B | 18.87 | 37.36/.0007% | 36.81/.001% | 20.82/28.6% | 28.6% | 6.83e2 |
| Pen 41-B | 21.85 | 38.07/.002% | X | X | | 2.14e2 |
| GS- CECUM | | | | | | |
| Pen 1-C | 18.04 | 37.13/.0005% | 23.28/4.18% | 20.11/26.5% | 30.7% | 2.68e2 |
| Pen 13-C | 21.29 | 37.51/.003% | 25.16/9.58% | 23.38/26.1% | 35.7% | 2.53e3 |
| Pen 2-N | 17.42 | 39.57/.00007% | 21.71/7.43% | 19.64/24.1% | 31.5% | 4.60e2 |
| Pen 14-N | 18.29 | 38.59/.0002% | 22.30/8.80% | 21.16/15.8% | 24.6% | X |
| Pen 6-B | 15.89 | X | 18.57/19.7% | 17.77/30.0% | 49.7% | X |
| Pen 41-B | 16.80 | 30.15/.02% | 22.47/3.22% | 18.55/32.5% | 35.74% | 3.27e4 |

### Example 17

### Use of FTA Cards and Diagnostic Reverse Transcriptase PCR to Determine Vibrio harveyi Hemolysin Toxin Gene Expression in Shrimp Pond Samples

The efficacy, stability, and yield of V. harveyi RNA extracted from cells preserved on Whatman FTA (fast technology for analysis) cards was determined using three RNA extraction protocols. Downstream performance was assessed with reverse transcription (RT)-qPCR, and the stability of samples stored between −20 and 37° C. was assessed after 20 days. This method was also used to detect changes in hemolysin (hly) toxin gene expression in cells exposed to varying pH and salinity treatments prior to storage on FTA cards. Two of the three RNA extraction protocols successfully recovered RNA from the FTA cards, and RNA yield did not decrease substantially after 20 days at −20, 25, or 37° C. RT-qPCR analysis of gene expression in the treatments at varying pH or salinity determined that hly gene expression increased up to five fold relative to control conditions. RT-qPCR protocols applied to FTA card samples collected in the field could be used to monitor for and reduce the incidence of vibriosis due to poor water quality concerns in aquaculture applications.

### Example 18

### Example of DNA Quality from Swab Sample Collection

Measurements were performed on a Quantus Fluorometer Machine and results are as follows:

Isolate 1=2.78 ng/μL

Isolate 2=2.81 ng/μL

Isolate 3=2.56 ng/μL

DNA Normalization Calculations

V₁C₁=V₂C Equation:

### Example 19

### PCR Amplification Conditions

Table 8 details the PCR conditions used to amplify the respective genes of interest for various microorganisms as described throughout the present disclosure.

TABLE 8
PCR Amplification Conditions

| | | | Primer | Anneal | | Primers | |
|---|---|---|---|---|---|---|---|
| Name | Species | Target | Conc | Temp | Ext Step | and Probes | Primers and Probe |
| diff 16s | C. difficile | 16s | 500-1000 | (50-70) | 77C 45 sec | Forward | GCAAGTTGAGCGATTTACCTTCGGT |
| | | | | 58C 20 | | | |
| | | | | sec | | | |
| | | | | | | Reverse | GTACTGGCTCACCTTTGATATTYAAGAG |
| | | | | | | Probe | FAM-TGCCTCCTCAAATATATTATCCCGT-TAMRA |
| tcd A | C. difficile | gene | 400 | 200 | 56C 50 sec | Forward | CAGTCGGATTGCAAGTAATTGACAAT |
| (HEX) | | | | | | Reverse | AGTAGTATCTACTACCATTAACAGTCTGC |
| set 2 | | | | | | Probe | HEX-TTGAGATGTAGCAGTGTCAGGAT-BHQ |
| perf 16s | C. perfinigens | 16s | 400 | 60C 30 | 72C 30 sec | Forward | TGAAAGATGGCATCATCATTCAAC |
| | | | | sec | | Reverse | GGTACCGTCATTATCTTCCCCAAA |
| cpa MP | C. perfinigens | gene | 300 | 200 | 57C 30 sec | Forward | GCTAATGTTACTGCCGTTGA |
| (HEX) | | | | | | Reverse | CCTCTGATACATCGTGTAAG |
| | | | | | | | HEX-TTGGAATCAAAACAAAGGATGGAAAAACTCAAG-TAMRA |
| Campy | Campylobacter | gene | 500 | 60C 30 | x | Forward | AGC AAA GGA TTT GGC GAT GC |
| cdt | | | | sec | | Reverse | TGC GTG ATT GCT TGC ATC AC |
| Campy 16s | Campylobacter | 16s | 500-1000 | (50- | 72C 45 sec | Forward | GGATGACACTTTTCGGAG |
| | | | | 70)61C | | Reverse | AATTCCATCTGCCTCTCC |
| | | | | 20 sec | | | |
| pcv2 | Circovirus | gene | 400 | 200 | 60C 40 sec | Forward | CGGATATTGTAkTCCTGGTCGTA |
| (FAM) | type 2 | | | | | Reverse | CCTGTCCTAGATTCCCCCTATTGATT |
| | | | | | | Probe | FAM-CTAGGCCTACGTGGTCTACATTTC-TAMRA |
| STb | E. coli | gene | 560 | 60C 20 | 72C 30 sec | Forward | TGCTAAACCAGTAGAGTCCTCAAAA |
| | | | | sec | | Reverse | GCAGGATTACAACACAATTCACAGC |
| Stx1 | E. coli | gene | 250 | 150 | 63C 30 sec | Forward | GTGGCATTAATACTGAATTGTCATC |
| (Texas | | | | | | Reverse | GCGTAATCCCACGGACTCTTC |
| Red) | | | | | | Probe | TxRed-TGATGAGTTTCCTTCTATGTGTCCCGGCAGAT-BHQ2 |
| Omp | H. parasuis | gene | 100, 400 | 100 | 58C 60 sec | Forward | TGATGGTCAATTGCGTCT |
| (FAM, | | | | | | Reverse | CGAGTCTCATAACGACCAAA |
| Cu5) | | | | | | Probe 1 | FAM-AATAATTCTCGTTTCGGTATTTCTATCAAACA-TAMRA |
| | | | | | | Probe 2 | Cys-AATAGTTCTCGTTTCGGTATTTCTATCAAACA-BHQ2 |
| hlyA146 | L. | gene | 1000 | 60 C 15 | 72C 1 min | Forward | AAATCTGTCTCAGGYGATGT |
| | monocytogenes | | | sec | | Reverse | CGATGATTTGAACTTCATCTTTTGC |
| iap | Listeria spp. | gene | 1000 | 60C 15 | 72C 1 min | Forward | cay CCGCWAGCACWG tag t |
| | | | | sec | | Reverse | GCGTCRACAGTWGTSCCHTT |
| Mhyo 46, | M. | gene | 500 | 300 | 60C 60 sec | Forward | ATTCCGATTGTTGCCTATGATC |
| (FAM) | hyopneumoniae | | | | | Reverse | AATTGAATCAAAAGCACCATCTTC |
| | | | | | | Probe | FAM_ATAGACCCGCCGCAAGTGAAAGAC-BHQ1 |
| | | | | | | Forward | CGCAAAGACTGAACCCACTAATTT |
| | | | | | | Reverse | TTGCCTCTGTTGTTACTTGGAGAT |
| PEDv | PED | gene | 0.5 | 0.5 | 60C 60 sec | Probe | Cy5-TGTTGCCATTGCCACGACTCCTGC-BHQ3 |
| (Cy5) | | | ul per | ul per | | | |
| sefA | S. | gene | 200 | 50 | 64C 60 sec | Forward | GGTAAAGGGGCTTCGGTATC |
| (Cy5) | Enteritidis | | | | | Reverse | TATTGGCTCCCTGAATACGC |
| | | | | | | Probe | Cy5-TGGTGGTGTAGCCACTGTCCCGT-BHQ1a |
| cps21 | S. suis | gene | 400 | 60C 30 | 72C 30 sec | Forward | GGTTACTTGCTACTTTTGATGGAAATT |
| (FAM) | | | | sec | | Reverse | CGCACCTCTTTTATCTCTTCCAA |
| | | | | | | Probe | FAM-TCAAGAATCTGAGCTGCAAAAGTGTCAAATTGA-TAMRA |
| sfc | S. | gene | 200 | 50 | 64C 50 sec | Forward | TGCAGAAAATTGATGCTGCT |
| (HEX) | Typhimurium | | | | | Reverse | TTGCCCAGGTTGGTAATAGC |
| | | | | | | Probe | JOE-ACCTGGGTGCGGTACAGAACCGT-BHQ1a |
| invA | Salmonella | gene | 400 | 65C 30 | 72 C 30 sec | Forward | CATTTCTATGTTCGTCATTCCATTACC |
| | spp. | | | sec | | Reverse | AGGAAACGTTGAAAAACTGAGGATTCT |
| ipaH | Shigello spp. | gene | 200 | 65 C 30 | 72C 30 sec | Forward | CGCGACGGACAACAGAATACACTCCATC |
| | | | | sec | | Reverse | ATGTTCAAAAGCATGCCATATCTGTG |
| A. fum-F | Aspergillus | | 200 | 60 C 30 | x | Forward | GCCCGCCGTTTCGAC |
| | fumigatus | | | sec | | | |
| A. fum-R | | | | | | Reverse | CCGTTGTTGAAAGTTTTAACTGATTAC |
| | | | | | | | Cy5-AATCAACTCAGACTGCACGCTTTCAGACAG-TAMRA |

1. A method of quantifying the expression level of a gene from a microorganism, the method comprising the steps of:
recovering the nucleic acid from a sample stabilized on a card,
amplifying the nucleic acid; and
quantifying the expression level of the gene, wherein a forward primer, and a reverse primer are used for the amplification.

2. The method of claim 1 further comprising the step of hybridizing a probe to the nucleic acid to specifically identify the gene.

3. The method of claim 1 wherein the reverse primer comprises a sequence selected from the group consisting of SEQ ID NO: 6 and SEQ ID NO: 8.

4. The method of claim 1 wherein the nucleic acid is DNA.

5. The method of claim 1 wherein the nucleic acid is RNA.

6. The method of claim 1 wherein the nucleic acid is amplified using PCR.

7. The method of claim 6 wherein the PCR is reverse transcription PCR.

8. The method of claim 6 wherein the PCR is reverse transcription-quantitative PCR.

9. The method of claim 2 wherein the probe is fluorescently labeled.

10. The method of claim 1 wherein the primer is fluorescently labeled.

11. The method of claim 1 wherein the microorganism is selected from the group consisting of Vibrio harveyi, Vibrio campbellii, Vibrio fluvialis, and Vibrio parahaemolyticus.

12. The method of claim 1 wherein the microorganism is selected from the group consisting of Clostridium perfringens, Campylobacter jejuni, and Campylobacter coli.

13. The method of claim 1 wherein the sample is a sample from an animal.

14. The method of claim 1 wherein the sample is an aquatic sample.

15. The method of claim 14 wherein the aquatic sample is from a fish hatchery.

16. The method of claim 14 wherein the aquatic sample is from a shrimp pond.

17. The method of claim 1 wherein the sample is an agricultural sample.

18. The method of claim 17 wherein the agricultural sample is from animal litter.

19. The method of claim 17 wherein the agricultural sample is a swab from a swine or a poultry species.

20. The method of claim 1 wherein the gene is a gene encoding a toxin.

21. The method of claim 1 wherein the gene is a gene of a bacterial species.

22. The method of claim 1 wherein the gene is a gene of a viral species.

23. The method of claim 1 wherein the gene is a hemolysin (hly) gene.

24. The method of claim 1 wherein the gene is a Clostridium perfringens enterotoxin (cpe) gene.

25. The method of claim 1 wherein the gene is a Clostridium perfringens beta toxin (cpb) gene.

26. The method of any-enema claim 1 wherein the nucleic acid is stabilized on the card for a period of time to allow transportation overseas.

27. The method of claim 1 wherein the nucleic acid is stabilized on the card for a period of time to allow transportation over greater than 1000 miles.

28. The method of claim 1 wherein the nucleic acid is stabilized on the card for a period of time to allow transportation over greater than 2000 miles.

29. The method of claim 1 wherein the nucleic acid is stabilized on the card for a period of time to allow transportation over greater than 3000 miles.

30. The method of claim 1 wherein the nucleic acid is stabilized on the card for a period of time to allow transportation over greater than 5000 miles.

31. The method of claim 1 wherein the card is a WHATMAN® FTA® Card.

32-43. (canceled)

44. The method of claim 1 wherein the microorganism is selected from the group consisting of swine enterotoxigenic E. coli (ETEC), avian pathogenic E. coli (APEC), attaching and effacing E. coli (EAEC), enterohaemorrhagic E. coli (EHEC), and shiga toxin-producing E. coli (STEC).

45. The method of claim 44 wherein the ETEC is an antigenic type selected from the group consisting of K88, F18, F41, 987P, and K99.

46. The method of claim 1 wherein reverse transcription-PCR and endpoint PCR are performed.

47. The method of claim 6, wherein the PCR is quantitative PCR.


SEQUENCE LISTING (69 sequences)

SEQ ID NO: 1 - DNA, 17 bp, Artificial Sequence
  Description of Artificial Sequence Synthetic primer
ctattggtggaacgcac

SEQ ID NO: 2 - DNA, 19 bp, Artificial Sequence
  Description of Artificial Sequence Synthetic primer
gtattctgtccatacaaac

SEQ ID NO: 3 - DNA, 19 bp, Artificial Sequence
  Description of Artificial Sequence Synthetic primer
gagttcggtttctttcaag

SEQ ID NO: 4 - DNA, 20 bp, Artificial Sequence
  Description of Artificial Sequence Synthetic primer
tgtagtttttcgctaatttc

SEQ ID NO: 5 - DNA, 17 bp, Artificial Sequence
  Description of Artificial Sequence Synthetic primer
ctattggtggaacgcac

SEQ ID NO: 6 - DNA, 19 bp, Artificial Sequence
  Description of Artificial Sequence Synthetic primer
cagcgaagtaggtaatgtc

SEQ ID NO: 7 - DNA, 19 bp, Artificial Sequence
  Description of Artificial Sequence Synthetic primer
gagttcggtttctttcaag

SEQ ID NO: 8 - DNA, 16 bp, Artificial Sequence
  Description of Artificial Sequence Synthetic primer
aaacggttatcggctg

SEQ ID NO: 9 - DNA, 20 bp, Artificial Sequence
  Description of Artificial Sequence Synthetic primer
ggcgtaaagcgcatgcaggt

SEQ ID NO: 10 - DNA, 22 bp, Artificial Sequence
  Description of Artificial Sequence Synthetic primer
gaaattctacccccctctacag

SEQ ID NO: 11 - DNA, 20 bp, Artificial Sequence
  Description of Artificial Sequence Synthetic primer
agagtttgatcmtggctcag

SEQ ID NO: 12 - DNA, 19 bp, Artificial Sequence
  Description of Artificial Sequence Synthetic primer
ggttaccttgttacgactt

SEQ ID NO: 13 - DNA, 39 bp, Artificial Sequence
  Description of Artificial Sequence Synthetic primer
gcggatccgcggccgctgcagagtttgatcctggctcag

SEQ ID NO: 14 - DNA, 38 bp, Artificial Sequence
  Description of Artificial Sequence Synthetic primer
gcggatcctctagactgcagtgccagcagccgcggtaa

SEQ ID NO: 15 - DNA, 37 bp, Artificial Sequence
  Description of Artificial Sequence Synthetic primer
ggctcgagcggccgcccgggttaccttgttacgactt

SEQ ID NO: 16 - DNA, 24 bp, Artificial Sequence
  Description of Artificial Sequence Synthetic primer
gcaagttgagcgatttacttcggt

SEQ ID NO: 17 - DNA, 28 bp, Artificial Sequence
  Description of Artificial Sequence Synthetic primer
gtactggctcacctttgatattyaagag

SEQ ID NO: 18 - DNA, 24 bp, Artificial Sequence
  Description of Artificial Sequence Synthetic probe
tgcctctcaaatatattatcccgt

SEQ ID NO: 19 - DNA, 26 bp, Artificial Sequence
  Description of Artificial Sequence Synthetic primer
cagtcggattgcaagtaattgacaat

SEQ ID NO: 20 - DNA, 29 bp, Artificial Sequence
  Description of Artificial Sequence Synthetic primer
agtagtatctactaccattaacagtctgc

SEQ ID NO: 21 - DNA, 24 bp, Artificial Sequence
  Description of Artificial Sequence Synthetic probe
ttgagatgatagcagtgtcaggat

SEQ ID NO: 22 - DNA, 24 bp, Artificial Sequence
  Description of Artificial Sequence Synthetic primer
tgaaagatggcatcatcattcaac

SEQ ID NO: 23 - DNA, 24 bp, Artificial Sequence
  Description of Artificial Sequence Synthetic primer
ggtaccgtcattatcttccccaaa

SEQ ID NO: 24 - DNA, 20 bp, Artificial Sequence
  Description of Artificial Sequence Synthetic primer
gctaatgttactgccgttga

SEQ ID NO: 25 - DNA, 20 bp, Artificial Sequence
  Description of Artificial Sequence Synthetic primer
cctctgatacatcgtgtaag

SEQ ID NO: 26 - DNA, 33 bp, Artificial Sequence
  Description of Artificial Sequence Synthetic probe
ttggaatcaaaacaaaggatggaaaaactcaag

SEQ ID NO: 27 - DNA, 20 bp, Artificial Sequence
  Description of Artificial Sequence Synthetic primer
agcaaaggatttggcgatgc

SEQ ID NO: 28 - DNA, 20 bp, Artificial Sequence
  Description of Artificial Sequence Synthetic primer
tgcgtgattgcttgcatcac

SEQ ID NO: 29 - DNA, 18 bp, Artificial Sequence
  Description of Artificial Sequence Synthetic primer
ggatgacacttttcggag

SEQ ID NO: 30 - DNA, 18 bp, Artificial Sequence
  Description of Artificial Sequence Synthetic primer
aattccatctgcctctcc

SEQ ID NO: 31 - DNA, 23 bp, Artificial Sequence
  Description of Artificial Sequence Synthetic primer
cggatattgtaktcctggtcgta

SEQ ID NO: 32 - DNA, 25 bp, Artificial Sequence
  Description of Artificial Sequence Synthetic primer
cctgtcctagattcccctattgatt

SEQ ID NO: 33 - DNA, 24 bp, Artificial Sequence
  Description of Artificial Sequence Synthetic probe
ctaggcctacgtggtctacatttc

SEQ ID NO: 34 - DNA, 25 bp, Artificial Sequence
  Description of Artificial Sequence Synthetic primer
tgctaaaccagtagagtcctcaaaa

SEQ ID NO: 35 - DNA, 25 bp, Artificial Sequence
  Description of Artificial Sequence Synthetic primer
gcaggattacaacacaattcacagc

SEQ ID NO: 36 - DNA, 25 bp, Artificial Sequence
  Description of Artificial Sequence Synthetic primer
gtggcattaatactgaattgtcatc

SEQ ID NO: 37 - DNA, 21 bp, Artificial Sequence
  Description of Artificial Sequence Synthetic primer
gcgtaatcccacggactcttc

SEQ ID NO: 38 - DNA, 31 bp, Artificial Sequence
  Description of Artificial Sequence Synthetic probe
tgatgagtttccttctatgtgtccggcagat

SEQ ID NO: 39 - DNA, 18 bp, Artificial Sequence
  Description of Artificial Sequence Synthetic primer
tgatggtcaattgcgtct

SEQ ID NO: 40 - DNA, 20 bp, Artificial Sequence
  Description of Artificial Sequence Synthetic primer
cgagtctcataacgaccaaa

SEQ ID NO: 41 - DNA, 32 bp, Artificial Sequence
  Description of Artificial Sequence Synthetic probe
aataattctcgtttcggtatttctatcaaaca

SEQ ID NO: 42 - DNA, 32 bp, Artificial Sequence
  Description of Artificial Sequence Synthetic probe
aatagttctcgtttcggtatttctatcaaaca

SEQ ID NO: 43 - DNA, 20 bp, Artificial Sequence
  Description of Artificial Sequence Synthetic primer
aaatctgtctcaggygatgt

SEQ ID NO: 44 - DNA, 25 bp, Artificial Sequence
  Description of Artificial Sequence Synthetic primer
cgatgatttgaacttcatcttttgc

SEQ ID NO: 45 - DNA, 22 bp, Artificial Sequence
  Description of Artificial Sequence Synthetic primer
cayccgcwagcacwgtagtagt

SEQ ID NO: 46 - DNA, 20 bp, Artificial Sequence
  Description of Artificial Sequence Synthetic primer
gcgtcracagtwgtscchtt

SEQ ID NO: 47 - DNA, 22 bp, Artificial Sequence
  Description of Artificial Sequence Synthetic primer
attccgattgttgcctatgatc

SEQ ID NO: 48 - DNA, 24 bp, Artificial Sequence
  Description of Artificial Sequence Synthetic primer
aattgaatcaaaagcaccatcttc

SEQ ID NO: 49 - DNA, 24 bp, Artificial Sequence
  Description of Artificial Sequence Synthetic probe
atagacccgccgcaagtgaaagac

SEQ ID NO: 50 - DNA, 24 bp, Artificial Sequence
  Description of Artificial Sequence Synthetic primer
cgcaaagactgaacccactaattt

SEQ ID NO: 51 - DNA, 24 bp, Artificial Sequence
  Description of Artificial Sequence Synthetic primer
ttgcctctgttgttacttggagat

SEQ ID NO: 52 - DNA, 24 bp, Artificial Sequence
  Description of Artificial Sequence Synthetic probe
tgttgccattgccacgactcctgc

SEQ ID NO: 53 - DNA, 20 bp, Artificial Sequence
  Description of Artificial Sequence Synthetic primer
ggtaaaggggcttcggtatc

SEQ ID NO: 54 - DNA, 20 bp, Artificial Sequence
  Description of Artificial Sequence Synthetic primer
tattggctccctgaatacgc

SEQ ID NO: 55 - DNA, 23 bp, Artificial Sequence
  Description of Artificial Sequence Synthetic probe
tggtggtgtagccactgtcccgt

SEQ ID NO: 56 - DNA, 27 bp, Artificial Sequence
  Description of Artificial Sequence Synthetic primer
ggttacttgctacttttgatggaaatt

SEQ ID NO: 57 - DNA, 23 bp, Artificial Sequence
  Description of Artificial Sequence Synthetic primer
cgcacctcttttatctcttccaa

SEQ ID NO: 58 - DNA, 33 bp, Artificial Sequence
  Description of Artificial Sequence Synthetic probe
tcaagaatctgagctgcaaaagtgtcaaattga

SEQ ID NO: 59 - DNA, 20 bp, Artificial Sequence
  Description of Artificial Sequence Synthetic primer
tgcagaaaattgatgctgct

SEQ ID NO: 60 - DNA, 20 bp, Artificial Sequence
  Description of Artificial Sequence Synthetic primer
ttgcccaggttggtaatagc

SEQ ID NO: 61 - DNA, 23 bp, Artificial Sequence
  Description of Artificial Sequence Synthetic probe
acctgggtgcggtacagaaccgt

SEQ ID NO: 62 - DNA, 27 bp, Artificial Sequence
  Description of Artificial Sequence Synthetic primer
catttctatgttcgtcattccattacc

SEQ ID NO: 63 - DNA, 27 bp, Artificial Sequence
  Description of Artificial Sequence Synthetic primer
aggaaacgttgaaaaactgaggattct

SEQ ID NO: 64 - DNA, 28 bp, Artificial Sequence
  Description of Artificial Sequence Synthetic primer
cgcgacggacaacagaatacactccatc

SEQ ID NO: 65 - DNA, 26 bp, Artificial Sequence
  Description of Artificial Sequence Synthetic primer
atgttcaaaagcatgccatatctgtg

SEQ ID NO: 66 - DNA, 15 bp, Artificial Sequence
  Description of Artificial Sequence Synthetic primer
gcccgccgtttcgac

SEQ ID NO: 67 - DNA, 27 bp, Artificial Sequence
  Description of Artificial Sequence Synthetic primer
ccgttgttgaaagttttaactgattac

SEQ ID NO: 68 - DNA, 30 bp, Artificial Sequence
  Description of Artificial Sequence Synthetic probe
aatcaactcagactgcacgctttcagacag

SEQ ID NO: 69 - DNA, 18 bp, Artificial Sequence
  Description of Artificial Sequence Synthetic primer
gagttcggtttctttcaa